SEQUENCE LISTING (11 sequences)

SEQ ID NO: 1 - protein, 15 aa, Artificial Sequence
  affinity peptide
QVSHWVSGLAEGSFG

SEQ ID NO: 2 - protein, 15 aa, Artificial Sequence
  synthetic affinity peptide 2
LSHTSGRVEGSVSLL

SEQ ID NO: 3 - protein, 12 aa, Artificial Sequence
  affinity peptide
CQMWAPQWGPDC

SEQ ID NO: 4 - protein, 17 aa, Artificial Sequence
  affinity peptide
CGSGSQLGPYELWELSH

SEQ ID NO: 5 - protein, 12 aa, Artificial Sequence
  affinity peptide
LLGPYELWELSH

SEQ ID NO: 6 - protein, 12 aa, Artificial Sequence
  affinity peptide
QLGPYELWELSH

SEQ ID NO: 7 - protein, 40 aa, Artificial Sequence
  affinity peptide
  DISULFID (5)..(18)
  DISULFID (9)..(26)
  DISULFID (29)..(38)
GPSLCHPECQPQNGSVTCFGPEADQCVACAHYKDPPFCVA

SEQ ID NO: 8 - protein, 30 aa, Artificial Sequence
  affinity peptide
  DISULFID (4)..(27)
VARCPSGVKPDLSYMPIWKFPDEEGACQPL

SEQ ID NO: 9 - protein, 11 aa, Artificial Sequence
  affinity peptide
VLPKTLCGGGS

SEQ ID NO: 10 - protein, 14 aa, Artificial Sequence
  affinity peptide
ACKYPLGHQCGGGS

SEQ ID NO: 11 - protein, 17 aa, Artificial Sequence
  affinity peptide
  DISULFID (1)..(12)
CQYNLSSRALKCGPGPG

(19) United States
(12) Patent Application Publication
(10) Pub. No.: US 2019/0160174 A1
(43) Pub. Date: May 30, 2019

(54) Methods and agents to treat tumor cells and cancer

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 16/271,877

(22) Filed: Feb. 10, 2019

Related U.S. Application Data
(63) Continuation-in-part of application No. 15/945,741, filed on Apr. 5, 2018.
(60) Provisional application No. 62/482,982, filed on Apr. 7, 2017.
(60) Provisional application No. 62/649,579, filed on Mar. 29, 2018.
(60) Provisional application No. 62/636,838, filed on Feb. 28, 2018.
(60) Provisional application No. 62/631,616, filed on Feb. 16, 2018.

(51) Int. Cl. A61K 47/54; C07K 16/22; A61K 47/44; A61K 9/00; C07K 16/26; C07K 16/28; A61P 35/00

(52) U.S. Cl. CPC A61K 47/544; C07K 16/22; A61K 47/44; A61K 47/555; A61K 38/00; C07K 16/26; C07K 16/2818; A61P 35/00; A61K 9/0019

(57) ABSTRACT

This disclosure provides cell surface anchoring conjugates, formulations comprising cell surface anchoring conjugates, TLR agonist and methods of using the same for boosting immunity in a subject and treating tumor cell and cancer.

### CROSS-REFERENCE TO RELATED APPLICATION

This application is a Continuation-In-Part application of U.S. application Ser. No. 15/945,741, filed on Apr. 5, 2018, which claims priority to U.S. Provisional Patent Application 62,482,982 filed on Apr. 7, 2017. It also claims priority to U.S. Provisional Patent Application 62,631,616 filed on Feb. 16, 2018, U.S. Provisional Patent Application 62,636,838 filed on Feb. 28, 2018 and U.S. Provisional Patent Application 62,649,579 filed on Mar. 29, 2018. The entire disclosure of the prior applications is considered to be part of the disclosure of the instant application and is hereby incorporated by reference.

### FIELD

This disclosure provides cell surface anchoring conjugates, formulations comprising cell surface anchoring conjugates, and methods of using the same for treating tumor cells and cancer. It also disclose method, composition and agent to boost immunity to treat tumor cells and cancer.

### BACKGROUND

The U.S. Food and Drug Administration has approved several checkpoint inhibitors for the treatment of various cancers over the last decade. Checkpoint inhibitors work by exposing cancer cells that have hidden from the immune system. Cancer cells deceive immune cells by sending signals at certain checkpoints that indicate they are not harmful. If not for these checkpoints, T-cells would attack healthy cells. Immunotherapy drugs disrupt the cancer cells' signals, exposing them to the immune system for attack. Researchers continue to search for new drugs, as well as combinations thereof with other known checkpoint inhibitor drugs, for use in treating tumors, as improved treatment results for patients with breast cancer, gastric cancer and other advanced cancers are needed.

### SUMMARY

The present disclosure is directed to compounds (agents), compositions and methods for treating cancer by treating and/or inhibiting tumors in a subject in need such as a cancer patient. The current invention relates to novel methods and agents to treat cancer. In some embodiments, the novel agents are in the form of antibody binding molecule-cell surface anchoring molecule conjugate that facilitates the lysis of cancer cells and/or antigen presenting using exogenous antibody. The antibody binding molecule-cell surface anchoring molecule conjugate that can enhance the killing of cancer cells and/or antigen presenting is called cancer cell inactivating agent. Also provided are pharmaceutical compositions comprising an antibody binding conjugate, such as, but not limited to, those described herein, and a Toll-like receptors (TLR) agonist. Suitable Toll-like receptors (TLR) agonists include, but are not limited to, CpG ODN (CpG oligodeoxynucleotide), polyinosinic:polycytidylic acid (poly IC), imiquimod, and the like, or a mixture thereof. In certain embodiments, the present disclosure is directed to a method of treating and/or inhibiting a tumor and its metastasis, comprising administering to a patient in need thereof a therapeutically effective amount of an antibody binding conjugate or a pharmaceutical composition as described herein.

### BRIEF DESCRIPTION OF THE DRAWINGS

FIG. 1 shows an example of a conjugate consisting of a 3β-cholesterylamine as cell surface anchoring molecule and Herceptin mimotope peptide and a short PEG as linker for cancer immunotherapy used in combination with immunity boosting agent

FIG. 2 illustrates the schema of using antibody binding molecule-optional linker-cell surface anchoring molecule conjugate to increase the antigen presenting and cancer cell killing

FIG. 3 shows examples of mimotope peptide based conjugate designs: mimotope peptide-(optional linker)-cholesterylamine conjugate

FIG. 4 shows examples of more than one unit of mimotope and more than one unit of cell surface anchoring molecule can be incorporated in the conjugate

FIG. 5 shows schema of soluble polymer backbone based conjugate

FIG. 6 shows an example of Herceptin mimotope peptide-cell membrane anchoring lipid molecule conjugate

FIG. 7 shows an example of Herceptin mimotope-membrane anchoring peptide conjugate

FIG. 8 shows examples of Herceptin mimotope peptide conjugate containing an NHS ester

FIG. 9 shows an example of DBCO labeled mimotope peptide

FIG. 10 shows examples of conjugate of antibody binding molecule-optional linker-affinity ligand for cancer cell surface molecule

FIG. 11 shows examples of Fc (or its fragment)-optional linker-cell surface anchoring molecule conjugate

FIG. 12 shows examples of cationic lipids

FIG. 13 shows an example of the construct of a JX-594 virus that can produce sialidase

FIG. 14 shows a construct of an anti-cancer bacterial that produce 3 proteins

FIG. 15 shows an example of steric hindrance based masking of sialidase to generate a sialidase prozyme.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an adjuvant” includes a plurality of adjuvants.

The term “about” when used before a numerical designation, e.g., temperature, time, amount, and concentration, including range, indicates approximations which may vary by (+) or (−) 30%, 15% or 5%.

As used herein, the term “treating” refers to preventing, curing, reversing, attenuating, alleviating, minimizing, inhibiting, suppressing and/or halting a disease or disorder, including one or more clinical symptoms thereof.

As used herein, the term “composition” refers to a preparation suitable for administration to an intended patient for therapeutic purposes that contains at least one pharmaceutically active ingredient, including any solid form thereof. In certain embodiments, the composition is formulated as an injectable formulation. In certain embodiments, the composition is formulated as a film, gel, or liquid solution.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue.

As used herein, the term “formulated” or “formulation” refers to the process in which different chemical substances, including one or more pharmaceutically active ingredients, are combined to produce a dosage form. In certain embodiments, two or more pharmaceutically active ingredients can be coformulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “delivery” refers to approaches, formulations, technologies, and systems for transporting a pharmaceutical composition in the body as needed to safely achieve its desired therapeutic effect. In some embodiments, an effective amount of the composition is formulated for intratumoral injection into the patient (e.g., intratumoral delivery).

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

As used herein, the term “pH buffering agent” refers to an aqueous buffer solution which resists changes in pH when small quantities of acid or base are added to it. pH Buffering solutions typically comprise of a mixture of weak acid and its conjugate base, or vice versa. For example, pH buffering solutions may comprise phosphates such as sodium phosphate, sodium dihydrogen phosphate, sodium dihydrogen phosphate dihydrate, disodium hydrogen phosphate, disodium hydrogen phosphate dodecahydrate, potassium phosphate, potassium dihydrogen phosphate and dipotassium hydrogen phosphate; boric acid and borates such as, sodium borate and potassium borate; citric acid and citrates such as sodium citrate and disodium citrate; acetates such as sodium acetate and potassium acetate; carbonates such as sodium carbonate and sodium hydrogen carbonate, etc. pH Adjusting agents can include, for example, acids such as hydrochloric acid, lactic acid, citric acid, phosphoric acid and acetic acid, and alkaline bases such as sodium hydroxide, potassium hydroxide, sodium carbonate and sodium hydrogen carbonate, etc. In some embodiments, the pH buffering agent is a phosphate buffered saline (PBS) solution (i.e., containing sodium phosphate, sodium chloride and in some formulations, potassium chloride and potassium phosphate).

In the current application the “/” mark means both “and” and “or”.

The current invention relates to novel methods, compositions and agents to treat tumor cell and cancer. In some embodiments, the novel agents/compounds are in the form of antibody binding molecule-cell surface anchoring molecule conjugate that facilitates the lysis of cancer cells and/or antigen presenting using exogenous antibody. The antibody binding molecule-cell surface anchoring molecule conjugate that can enhance the killing of tumor/cancer cells and/or antigen presenting is called cancer cell inactivating agent. The said cancer cell inactivating agent can be injected intratumorally to treat cancer. The conjugate can further comprise a cancer cell binding moiety to increase its targeting to cancer cell, which will allow intravenous (iv) injection or intramuscular (IM) or subcutaneous (SC) instead of intratumoural injection.

The current invention also discloses methods to treat tumor cell and cancer and to boost immunity against tumor cell. The method comprises giving patient said cancer cell inactivating agent and/or agent can enhance cancer cell antigen presenting or in combination with an immune activity enhancing agent (immunity boosting agent) and exogenous antibody that can bind with the cancer cell inactivating agent. In addition to above treatment regiment, immune checkpoint inhibitors at therapeutical effective amount could be given to further enhance this treatment. In some embodiments, the immune activity enhancing agent (immunity boosting agent) is also called vaccine adjuvant type agent. In some embodiments the immune activity enhancing agent is given by intratumoural injection. It can be given to the patient by intratumoural injection as a mixture with the said cancer cell inactivating agent/agent can enhance cancer cell antigen presenting or sequentially (before or after) to the same tumor injected with the cancer cell inactivating agent. For example, a solution formulation containing both said cancer cell inactivating agent and/or agent that can enhance cancer cell antigen presenting and immunity boosting agent can be injected into the tumor at 50 uL˜1000 uL/cm3 tumor volume. Suitable tumor can be any type of solid tumor as long as it allows intratumoral injection. The antibody that can bind with the cancer cell inactivating agent and/or agent can enhance cancer cell antigen presenting can be given by intratumoural injection (e.g. 5˜50 mg/cm3 tumor volume) or be given systematically at the same time or within ±3 weeks. Examples of these antibody are recombinant therapeutic antibodies used for cancer treatment. The antibody can be given by intratumoural injection including IV, IM and SC injection together with the immune activity enhancing agent.

The disclosure also relates to methods of treating cancer. Accordingly, provided herein is a method of treating and/or inhibiting a solid tumor, comprising administering to a patient in need thereof a therapeutically effective amount of the cell surface anchoring conjugate, a formulation or pharmaceutical composition as described herein. The cell surface anchoring conjugate, a formulation or pharmaceutical composition as described herein can be injected intratumorally to treat the cancer. In certain embodiments, the cell surface anchoring conjugate, a formulation or pharmaceutical composition further comprises a cancer cell binding domain to increase its targeting to cancer cell, which will allow intravenous (IV) injection instead of intratumoral injection. In certain embodiments, the treating and/or inhibiting comprises preventing metastasis of the tumor. In other embodiments, the method comprises administering a therapeutically effective amount of an immune check point inhibitor, such as T lymphocyte antigen 4 (CTLA4) blocking antibody, PD-1 blocking antibody, PD-L1 blocking antibody, ipilimumab, tremelimumab, atezolizumab, nivolumab or pembrolizumab, or a combination thereof.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 100 mg/kg; with levels in the range of about 0.05 up to 10 mg/kg are generally applicable. A therapeutically effective dose can be estimated initially from cell culture assays by determining an IC₅₀. A dose can then be formulated in animal models to achieve the IC₅₀ as determined in cell culture. Such information can be used to more accurately determine useful initial doses in humans. Levels of drug in plasma or tumor may be measured, for example, by HPLC.

Also provided are methods of inhibiting or eliminating cancer cells in a tumor and/or preventing metastasis. The method comprises administering to a patient in need thereof a formulation or composition as described herein, which comprises a cancer cell lysing agent, such as cell surface anchoring conjugate, in combination with an immune function enhancing agent. The composition may be administered via intratumoral injection to the tumor. The immune function enhancing agent can be given to the patient by intratumoral injection as a mixture with the cancer cell lysing agent, such as a cell surface anchoring antigen conjugate, or sequentially (before or after) to the same tumor injected with a cancer cell lysing agent. For example, a liquid formulation containing both a cancer cell lysing agent and an immune function enhancing agent can be injected into the tumor (e.g., at 50 μL to about 1,000 μL/cm³ tumor volume. The tumor be any type of solid tumor, provided it allows intratumoral injection.

In summary, provided are methods to kill cancers cells in a tumor and/or to prevent or delay metastasis by treating a primary tumor. The method comprises administering to a patient in need thereof, a cancer cell lysing agent optionally in combination with an immune function enhancing agent. Immune checkpoint inhibitors at therapeutically effective amounts can also be administered to further enhance this treatment. The immune function enhancing agent is administered by intratumoral injection to the primary tumor. It can be administered to a subject in need thereof by intratumoral injection as a mixture with a cancer cell lysing agent or sequentially (before or after) to the same tumor injected with the cancer cell lysing reagent. The treatment to the primary tumor will induce an immune response against distant and secondary tumor to kill the cancer cells within, as well as prevent the metastasis of tumor. The composition used for intratumoral injection comprises a cancer cell lysing agent and an immune function enhancing agent in a pharmaceutical acceptable carrier. The formulation comprises a cancer cell lysing agent and an immune function enhancing agent in a pharmaceutical acceptable carrier. It can be injectable liquid or solid dosage form, such as a lyophilized formulation, that can be reconstituted with an injectable liquid. The cancer cell lysing agent and immune function enhancing agent can be in the form of an active drug, prodrug, liposome, micelle, emulsion, gel, implant, thermal phase changing formulation, insoluble precipitate (e.g. in complex with condensing reagent), conjugated to polymer drug carrier (e.g. dextran), coated on the surface or encapsulated within biodegradable micro particle or nanoparticle. A thermal phase changing formulation is a formulation that changes its phase from a liquid to a semisolid when the temperature increases. Such formulations typically use poloxamer as an excipient. Exemplary sizes of the microparticles or nanoparticles is between 10 nm and 100 μm.

The current invention also discloses novel compositions/formulations to treat tumor cell and cancer and to boost immunity. The compositions/formulation comprises said cancer cell inactivating agent and/or agent can enhance cancer cell antigen presenting and immune activity enhancing agent in a pharmaceutical acceptable carrier. It can be injectable solution or solid dosage form such as lyophilized formulation that can be reconstituted to injectable solution. The formulation contains cancer cell inactivating agent and/or agent can enhance cancer cell antigen presenting and immune activity enhancing agent as well as pharmaceutical acceptable excipients suitable for injection such as buffering salt (e.g. PBS salt), amino acid, carbohydrate (e.g. mannose, trehalose) and surfactant (e.g. PEG, tween, PVA, lethicin) or their combination. The formulation can further comprise antibody that can bind with the cancer cell inactivating agent/agent can enhance cancer cell antigen presenting.

The current invention also discloses methods to boost immunity and kill cancers cells in distant tumor and/or prevent metastasis. The method comprises giving the object in need the said cancer cell inactivating agent and/or agent can enhance cancer cell antigen presenting and/or in combination with an immune activity enhancing agent. The antibody that can bind with the cancer cell inactivating agent/agent can enhance cancer cell antigen presenting is given by intratumoural injection (e.g. 0.5˜50 mg/cm3 tumor volume) or be given systematically at the same time or within ±3 weeks. In addition to above treatment regiment, immune checkpoint inhibitors at therapeutical effective amount could be given to further enhance this treatment. The immune activity enhancing agent is given by intratumoural injection to the primary tumor. It can be given to the object in need by intratumoural injection as a mixture with the said cancer cell inactivating agent and/or agent can enhance cancer cell antigen presenting or sequentially (before or after) to the same tumor injected with the cancer cell inactivating agent. The injected cancer cell inactivating agent and/or agent can enhance cancer cell antigen presenting will be present on the cancer cell surface and attract the antibody added. The antibody will produce cancel cell killing effect and/or cancer antigen presenting to immune cells, therefore generate immune response against cancer cells. The treatment to the primary tumor will induce immune response against distant and secondary tumor to kill the cancer cells within, as well as prevent the metastasis of tumor.

Examples of suitable immune check point inhibitors include PD-1 antagonist such as antibody against PD-1, antibody against PD-L1, antibody against CTLA-4, antibody against OX40 or other OX40 agonist, or their combinations. Some are commercial available and can be readily used for the current invention such as Ipilimumab, Tremelimumab, Atezolizumab, Nivolumab and Pembrolizumab. They can be administered to the patient after the cancer cell inactivating agent treatment. For example, the patient can be intravenously injected with Ipilimumab 3˜10 mg/kg every 3 weeks for 4 doses after treatment or Atezolizumab 1200 mg IV q3wk after treatment until disease progression. The current treatment dosing of these immune check point inhibitors can be used. They can be also be injected intratumorally or injected proximal to the tumor draining lymph node, where lower than systematic amount can be used. They can be co-formulated with the above cancer cell inactivating agent and/or agent can enhance cancer cell antigen presenting and immune activity enhancing agent; and used as intratumoral injection.

Examples of suitable immune function enhancing agent include pattern recognition receptor (PRR) ligands, RIG-I-Like receptor (RLR) ligands, Nod-Like receptor (NLR) ligands, C-Type Lectin Receptors (CLR) ligands, and Toll-like receptor ligands such as a TLR3 ligand, a TLR4 ligand, a TLRS ligand, a TLR7/8 ligand, a TLR9 ligand, or a combination thereof. The immune function enhancing agent can be a vaccine adjuvant. Preferably the Toll-like receptor ligand is a Toll-like receptors (TLR) agonist. Examples of suitable immune activity enhancing agent (immunity boosting agent) include PRR Ligand, TLR3 Ligand, RLR Ligand, TLR4 Ligand, TLR5 Ligand, TLR7/8 Ligand, TLR9 Ligand, NOD2 Ligand, interleukin 12, tumour necrosis factor, interferon gamma (IFNγ), immunomodulatory imide drugs (IMiDs such as thalidomide, lenalidomide and pomalidomide, Treg inhibitory agent such as inhibitory antibody against Treg or their combinations. Many of them are commercial available (e.g. those listed in invivogen) and can be readily used for the current invention. Example includes imidazoquinoline family of TLR7/8 Ligands (e.g. imiquimod(R837), gardiquimod, resiquimod (R848), 3M-052, 3M-852, 3M-S-34240), CpG ODNs such as ODN 1826 and ODN 2216, TLR agonist including TLR peptide agonist disclosed in patent applications WO2018055060A1, WO2013120073A1, WO2016146143A1 and US20180133295A1 and their citations, synthetic analogs of dsRNA, such as poly IC (e.g. Poly ICLC, polylC-Kanamycin, PolyI:PolyC12U), TLR4/5 Ligands such as Bacterial lipopolysaccharides (LPS, e.g. monophosphoryl lipid A), bacterial flagellin (e.g. Vibrio vulnificus flagellin B), Glucopyranosyl lipid A (GLA), TLR7 agonist Loxoribine or their derivatives/analogues, or their combinations. They can be in form of active drug, prodrug, liposome, emulsion, micelle, insoluble precipitate (e.g. in complex with condensing agent), conjugated to polymer drug carrier (e.g. dextran) or encapsulated in biodegradable micro particle/nano particle (e.g. those made of biodegradable polymer such as PLA, PLGA, PCL, PGA or PHB). The use and preparation of vaccine adjuvant encapsulated micro particle/nano particle or its prodrug are well known to the skilled in the art. Examples of them suitable for the current invention can be found in or adopted from U.S. patent application Ser. No. 13/560,955, U.S. Ser. No. 12/764,569, U.S. Ser. No. 12/788,266, publication in Vaccine. 2014 May 19; 32(24):2882-95, Science. 2015 Jun. 19; 348(6241): aaa8205 and Nat Commun. 2016; 7: 13193. And their related citations. In one example, PLGA-R837 (R837 encapsulated in Poly Lactide-co-Glycolide particles) nanoparticle are prepared using o/w single-emulsion method. Briefly, R837 (TLR7 ligand) is dissolved in DMSO at 2.5 mg/ml. A total of 50 μL R837 is added to 1 ml PLGA (5 mg/ml) dissolved in dichloromethane. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R837 nanoparticles are obtained after centrifugation at 3,500 g for 20 min. Combination of vaccine adjuvant (immune activity enhancing agent) and cancer cell inactivating agent can also be encapsulated together in micro/nano particles. For example, R837 or R848 is dissolved in DMSO at 2.5 mg/ml. A cancer cell inactivating agent of the current invention is dissolved in DMSO at 50 mg/ml. 50 μR837/R848 and 50 μcancer cell inactivating agent solutions in DMSO are added to 1 ml mPEG-PLGA (10 mg/ml) dissolved in acetonitrile. Next, the mixture was dropwise added into 5 ml water containing 100 mg poly IC. After 1 h stirring and 12 h standing, the nanoparticles are obtained after centrifugation at 22,000 g for 5 min.

Preferably the immune activity enhancing agent (immunity boosting agent) is given intratumorally at therapeutical effective amount. For example, the imiquimod can be given at the amount between 100 ug˜100 mg as free drug or given as 10 mg˜1000 mg micro or nano particle encapsulating 1 mg˜100 mg imiquimod. Other suitable dosing can be used, as long as it can produce satisfactory therapeutical effect, which can be determined experimentally by screening and testing with well-known protocol and methods.

In some embodiments, the principle of cancer cell inactivating agent/agent that can enhance cancer cell antigen presenting in the current invention is to direct antibody or cytotoxic T cell to cancer cells, releasing tumor antigen for cancer immunotherapy. It will form in situ cancer vaccine and promote strong immune response with the locally injected immune activity enhancing agent.

It has the general structure as following, which is also celled cell surface anchoring conjugate:

antibody binding molecule-optional linker-cell surface anchoring molecule conjugate

In some embodiments, the cell surface anchoring molecule is cell membrane anchoring molecule, therefore the general structure of the conjugate is:

antibody binding molecule-optional linker-cell membrane anchoring molecule conjugate

The antibody binding molecule can be the antigen of endogenous antibody in patient or the antigen of exogenous antibody given to the patient. Examples of exogenous antibody is the recombinant therapeutic antibody used for cancer treatment. The antigen can be the biopolymer (e.g. protein or its fragment) or peptide or small molecule used to induce/screen the antibody. It can be the epitope or mimotope of the antibody.

The antibody binding molecule can be affinity ligand for antibody other than antigen. It can be aptamer that can bind with antibody, antibody mimetic that can bind with antibody, a second antibody or antibody fragment that can bind with endogenous or exogenous antibody (e.g. a mouse antibody or Fab against human antibody's Fc region or human antibody's Fab region).

Preferably the binding of the said ligand will not inhibit antibody's complement activation activity and/or antigen presenting effect induced by antibody binding.

For example, when the exogenous antibody is Herceptin (Trastuzumab), the antibody binding molecule can be HER2/neu receptor or its derivatives or fragment such as Recombinant Human ErbB2/Her2 Fc Chimera Protein (e.g. R&D system, #1129-ER-050), Human HER2/ErbB2 Protein with His Tag (e.g. Sino Biological #10004-H08H-50) and ErbB2 (e.g. Thermo Fisher # PV3366HER2).

Trastuzumab binds to domain IV of the extracellular segment of the HER2/neu receptor. Therefore, antibody binding molecule can be domain IV of the extracellular segment of the HER2 instead of the full length HER2.

In some embodiments, the conjugate comprises a mimotope therefore is called cell surface anchoring mimotope antigen conjugate. For example, the antibody binding molecule can also be the mimotope of Herceptin (Trastuzumab). Examples of mimotope include those described in J Immunol. 2007 Jun. 1; 178(11):7120-31. J Immunol. 2004 Jul. 1; 173(1):394-401. Mol Immunol. 2005 May; 42(9):1121-4. J Biol Chem. 2005 Feb. 11; 280(6):4656-62. Anal Chem. 2011 Dec. 1; 83(23): 8928-8936. Oncoimmunology. 2016 Apr. 21; 5(7):e1171446. And the E75 synthetic peptide used in NeuVax.

Examples of the mimotope are (H₂N— means the peptide starts with a free N terminal, —COOH means the peptide ends with a free COOH terminal,—means the linking(conjugate) site:

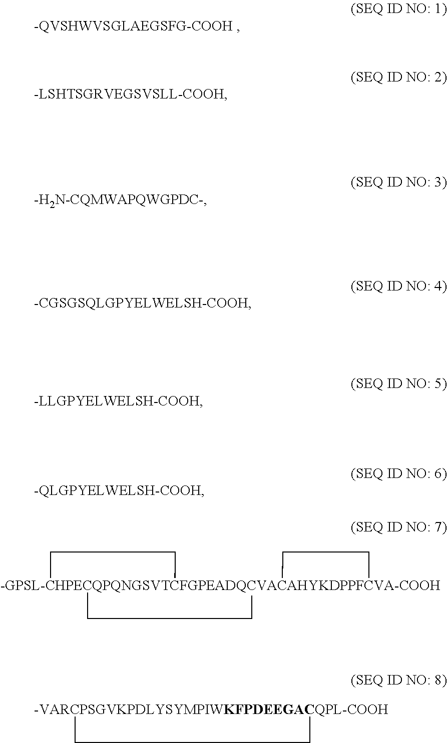

The mimotope that can be used in current invention is not limited to peptide and macro molecule. It can also be non-peptide structure based agent such as small molecule based or polynucleotide based agent as long as it can bind with the antibody selectively with high affinity.

In another example, when the exogenous antibody is Cetuximab, the antibody binding molecule can be epidermal growth factor receptor or its fragments or derivatives such as Recombinant Human EGF Protein (e.g. R&D system, #236-EG-01M, #4289-EG-025 or Prospec # Sf9 PKA-344).

Cetuximab binds to epidermal growth factor receptor (EGFR), targeting the extracellular domain of the epidermal growth factor receptor (EGFR). Its conformational epitope recognized by cetuximab covers a large surface on domain III of the EGFR. Therefore, antibody binding molecule can be extracellular domain or domain III of the EGFR instead of the full-length EGFR.

The antibody binding molecule can also be the mimotope of Cetuximab. For example, those described in J Natl Cancer Inst. 2005 Nov. 16; 97(22):1663-70. Oncogene (2010) 29, 4517-4527. And Neoplasia. 2012 Nov.; 14(11):1023-1031. Exemplary sequence of the mimotope include VLPKTLCGGGS-(SEQ ID NO: 9) or ACKYPLGHQCGGGS-(SEQ ID NO: 10) or cyclic C-QYNLSSRALK-C-GPGPG-(SEQ ID NO: 11).

Similarly, other antibody drug including bi-specific antibody, tri-specific antibody and antibody-drug conjugate targeting cancer cell and their antibody binding molecule (e.g. epitope or mimotope) can also be used, such as Panitumumab, Zalutumumab, zalutumumab, nimotuzumab, matuzumab, Pertuzumab, margetuximab, Bevacizumab, Brentuximab, Ado-trastuzumab emtansine, Catumaxomab and Blinatumomab.

U.S. patent application Ser. No. 15/945,741 by the current inventor disclosed native antigen-optional linker-cell surface anchoring molecule conjugate for cancer treatment. The native antigen in the disclosure and embodiments of said prior application can be replaced with affinity ligand such as antigen for the exogenous antibody given to the patient, which results in the antibody binding molecule-optional linker-cell surface anchoring molecule conjugate of the current invention. The antigen for the exogenous antibody can be the biopolymer (e.g. protein or its fragment) or peptide or small molecule used to induce/screen the antibody. It can be the epitope or mimotope of the exogenous antibody.

The conjugate molecule can contain one or more antigens as well as combinations of different antigens. An optionally linker or spacer (e.g. a short peptide or short PEG with MW<1500) can be used to connect the antigen and cell membrane anchoring molecule. The linker can contain one or more Lys or Arg or other positively charged group to increase its affinity to cell membrane. The amine of the cholesterylamine in the conjugate can be converted to quaternary ammonium if the cell membrane anchoring molecule is cholesterylamine. The different moieties (antibody binding molecule, optional linker and cell surface anchoring molecule) in the conjugate are jointed together by covalent bond such as amide bond, amine bond and ether bond, which are widely used in bio conjugation chemistry well known to the skilled in the art.

Several methods and cell surface anchoring molecule can be used to anchor antigen to cell surface, including covalent attachment to membrane proteins using reactive molecules as cell surface anchoring molecule (e.g. maleimide containing molecules to react with —SH of cell surface proteins, NHS ester containing molecules to react with amine group at cell surface, aldehyde containing molecules to react with cell surface molecules), modification of cell surface glycoproteins through oligosaccharide biosynthesis (e.g. using metabolic cell-labeling to introduce azide group on cell surface and then conjugate antigen with it using click chemistry such as those described in Nature Chemical Biology volume 13, pages 415-424 and Nature. 2004 Aug. 19; 430(7002):873-7.) and hydrophobic anchoring to the cell membrane using hydrophobic molecules as cell surface anchoring molecule. Examples of them can be found in Bioconjugate Chem., 2014,25 (12), p 2134-2143.

Practically, the process of hydrophobic anchoring simply involves mixing the hydrophobic anchors with cells, which allows for the spontaneous transfer of the anchors from the solution phase to the outer leaflet of the plasma membrane.

To increase the duration of the anchors on the cell membrane because of dissociation processes or endocytotic disappearance from the cell membrane, two approaches can be used, including increasing the number of hydrophobic anchoring groups, and extending the alkyl chain length of the anchoring groups. For example, polymer-based anchors bearing multiple hydrophobic anchoring units along the main hydrophilic polymer can be used to prolong the longevity of the anchor on the cell surface. The cell membrane anchoring molecule can be hydrophobic molecules such as lipid or cell membrane anchoring peptide, which can be found in many publications (e.g. Bioconjugate Chem., 2014, 25 (12), p 2134-2143).

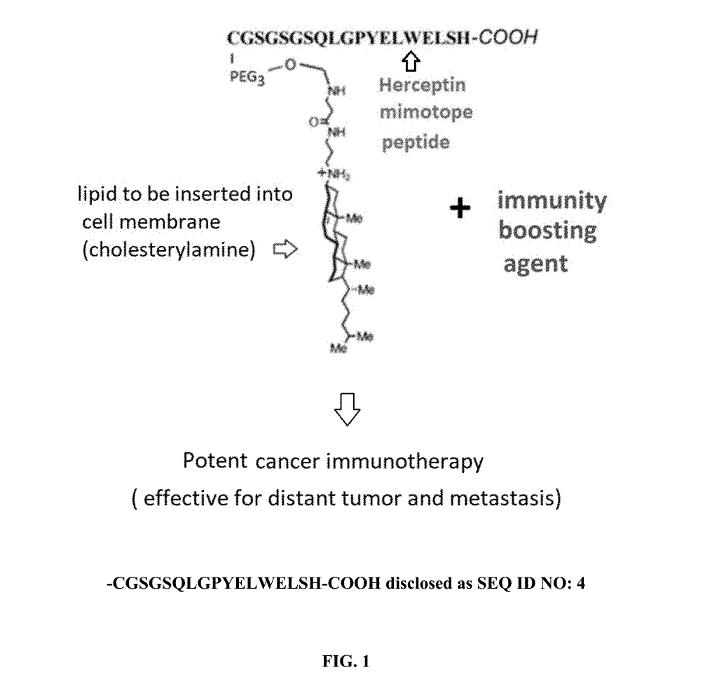

The example in FIG. 1 shows the conjugate consisting of a 3β-cholesterylamine as cell surface anchoring molecule and Herceptin mimotope peptide and a short PEG as linker, to increase its potency. It can target none or low HER2+expression tumor. The immunity boosting agent can be co-injected intratumorally to turn the tumor into a in situ vaccine.

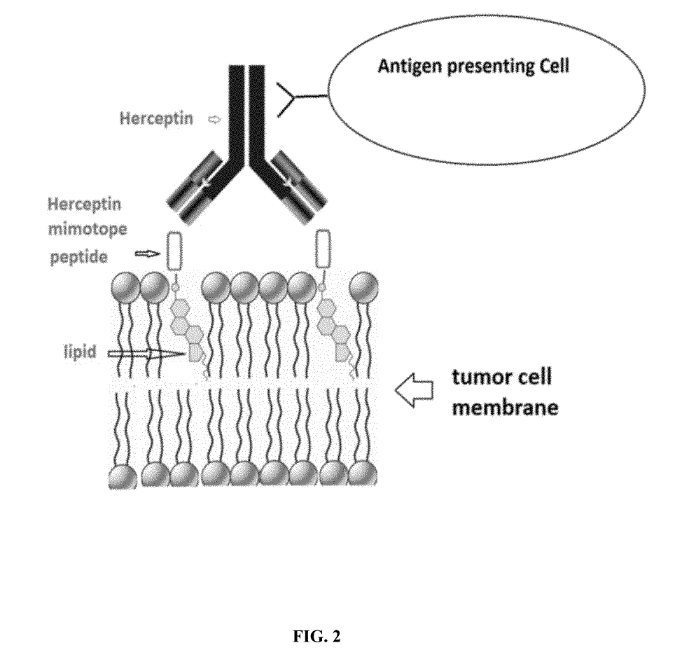

FIG. 2 below illustrates the mechanism of an example of using antibody binding molecule-optional linker-cell surface anchoring molecule conjugate to increase the antigen presenting and cancer cell killing, wherein the cell surface anchoring molecule is a lipid type molecule that can insert into cancer cell membrane and the antibody binding molecule is Herceptin mimotope peptide and the exogenous antibody is Herceptin. The Herceptin can be injected either intratumorally or injected systematically. It works as an artificial expression of antigen by introducing an antibody epitope to the tumor cell surface. The introduced antibody epitope will allow injected antibody drug to target these tumor cells, which improve the antigen presenting by APC. Further in combination with immunity boosting agent as a cancer immune therapy strategy will improve the efficacy and application of current oncology antibody drugs. The lipid in the conjugate can be replaced with other cell surface immobilizing molecules.

In some embodiments, the preferred cell membrane anchoring molecule for the conjugate is fatty acid or long alkyl chain or 3β-cholesterylamine or its analogues or derivatives, 3β-cholesterylamine type molecule enables endosome recycling of conjugate for long cell surface anchoring half-life. It can be either in monomer or dimer or trimer or oligomer format within the conjugate. The antibody binding molecule can also be either in monomer or dimer or trimer or oligomer format within the conjugate.

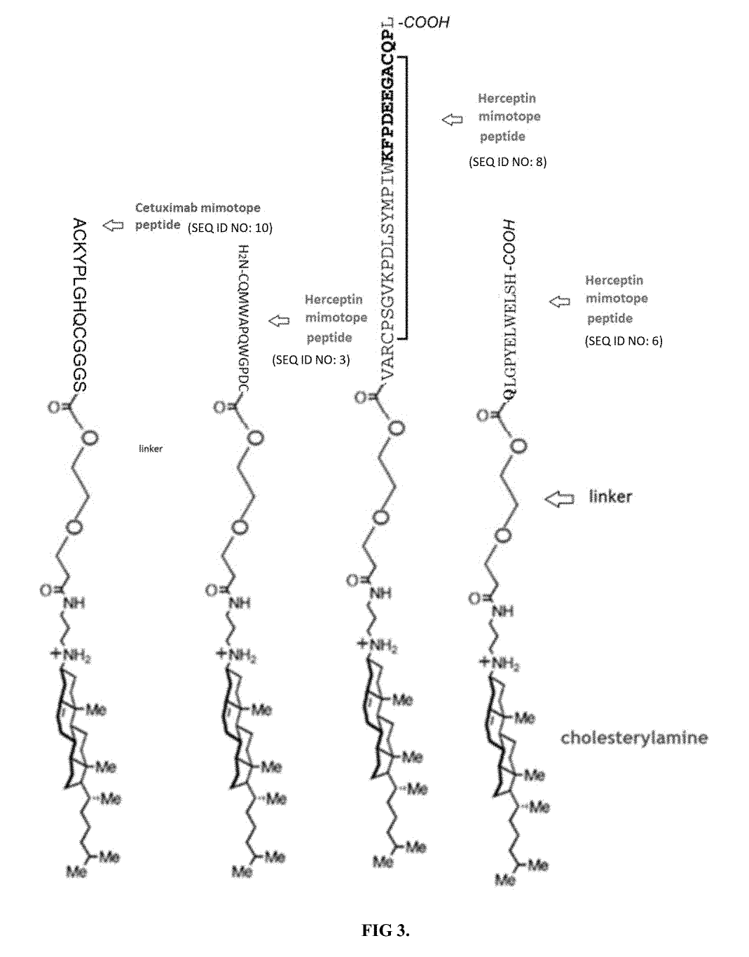

Examples of 3β-cholesterylamine, 3β-cholesterylamine containing moiety and their derivatives that can be used for the conjugate can be found in U.S. patent application Ser. No. 15/945,741. For example, FIG. 3 of U.S. patent application Ser. No. 15/945,741 shows examples of 3β-cholesterylamine, 3β-cholesterylamine containing moiety and their derivatives used for the conjugate. In the figure, the amine group can be substituted with linear or branched alkyl group or alkenyl group or alkynyl or aryl group containing 1 to 30 carbons such as methyl, ethyl or other low alky groups (R. R1, R2 in the figure). The 3β-cholesterylamine can be further conjugated with a positive charge group containing moiety such as an arginine in the figure. The double bond alkenyl —C═C-group in the cholesterylamine can be replaced with a saturated alkyl —C—C-group, therefore become a cholestane derivative. In some preferred embodiments, the general structure of the cell membrane anchoring molecule is 3-amine group substituted triterpenes including cholestane, cholestadiene and cholestane. The 3-amine group can be either alpha or beta configuration. In other preferred embodiments, the general structure of the cell membrane anchoring molecule is cationic lipid where the conjugation is at the cationic end containing secondary, tertiary or quaternary amine group. For example, FIG. 6 of U.S. patent application Ser. No. 15/945,741 shows additional examples of cell membrane anchoring molecule/moiety.

Exemplary structures of the conjugate include Herceptin mimotope-cholesterylamine, Cetuximab mimotope-cholesterylamine, Herceptin mimotope-linker-cholesterylamine, Cetuximab mimotope-linker-cholesterylamine, Cetuximab mimotope oligomer-linker(optional)-cholesterylamine, Herceptin mimotope oligomer-linker(optional)-cholesterylamine, Herceptin mimotope-linker-cholesterylamine-Cetuximab mimotope.

FIG. 3 shows examples of the mimotope peptide based conjugate design: mimotope peptide-(optional linker)-cholesterylamine conjugate, which will allow it bind with antibody and therefore eliminate the anchored cells and improve tumor antigen presenting. Short PEG is used as linker in them.

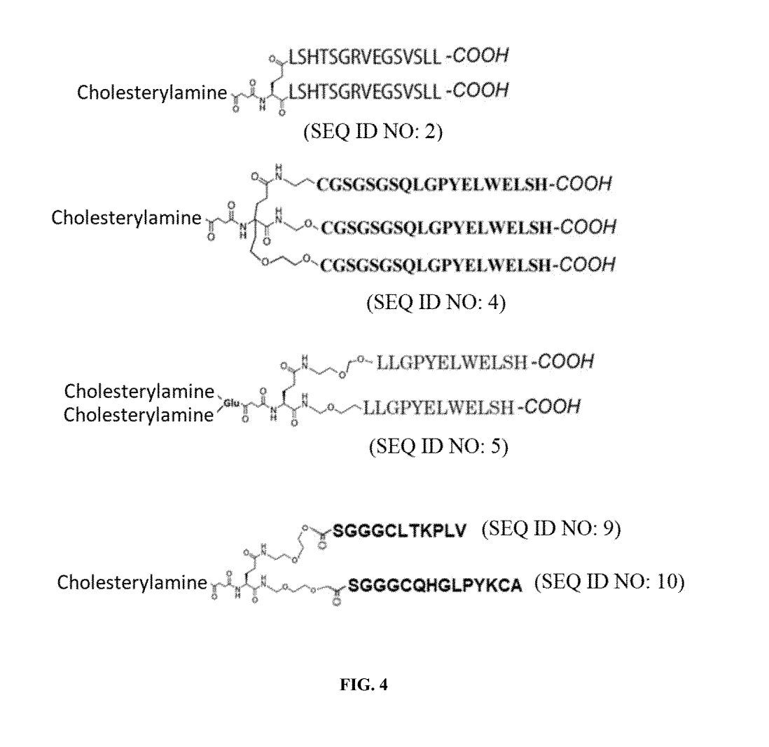

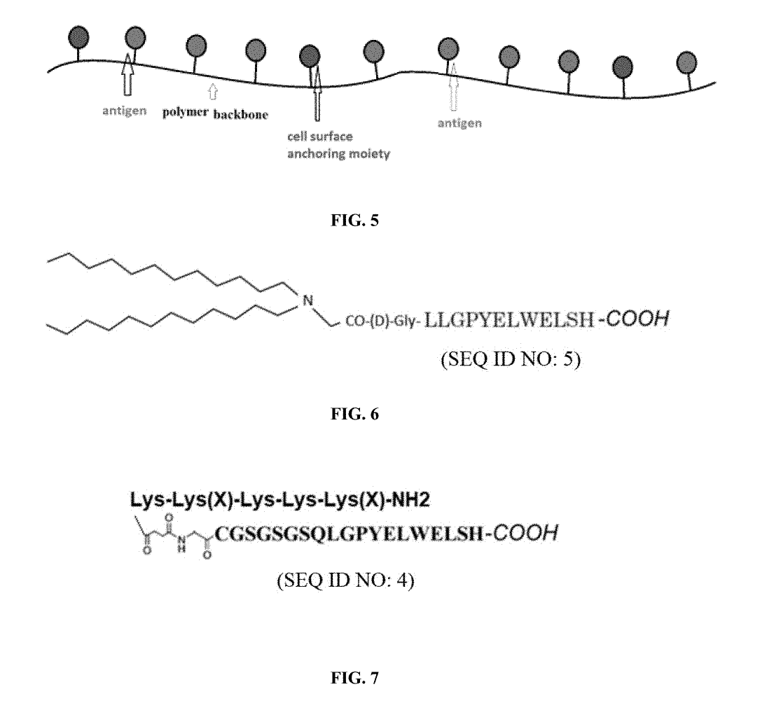

More than one unit of antigen (e.g. mimotope) or affinity ligand for antibody, more than one type of antigen (e.g. mimotope) or affinity ligand for antibody and more than one unit of cell surface anchoring molecule such as cholesterylamine can be incorporated in the conjugate as shown in FIG. 4. They can also be conjugated to a soluble polymer backbone (e.g. dextran, poly peptide, poly acrylic acid or the like) as shown in FIG. 5. In soluble polymer back bone can also be used, which is essentially a nano or micro particle. The cell membrane/surface anchoring molecule can also be molecule other than cholesterylamine, such as lipid molecule and cell membrane anchoring peptide. Example of the lipid molecule suitable for the current invention include fatty acid or its derivative, phospholipid glycerolipid, glycerophospholipid, sphingolipid, ceramide, glycerophosphoethanolamine, sterol or steroid. As described previously, besides 3β-cholesterylamine, other cell membrane anchoring lipid molecules can also be used. Example of Herceptin mimotope peptide-lipid conjugate is shown in FIG. 6.

Cell membrane anchoring molecule can also be cell membrane anchoring peptide, for example, those described in Bioconjugate Chem., 2014, 25 (12), 2134-2143. For example, Cetuximab mimotope-membrane anchoring peptide conjugate has the structure:

cyclic C-QYNLSSRALK-C-GPGPG-Lys-Lys(X)-Lys-Lys-Lys(X)—NH₂ (Lys(X): N-ε-palmitoyl-L-lysine, cyclic C-QYNLSSRALK-C-GPGPG-disclosed as SEQ ID NO: 11, C—C cyclization by —S—S—bond)

In another example, Herceptin mimotope-membrane anchoring peptide conjugate has the structure shown in FIG. 7 (Lys(X): N-ε-palmitoyl-L-lysine, the linker conjugate to the N terminal of Lys).

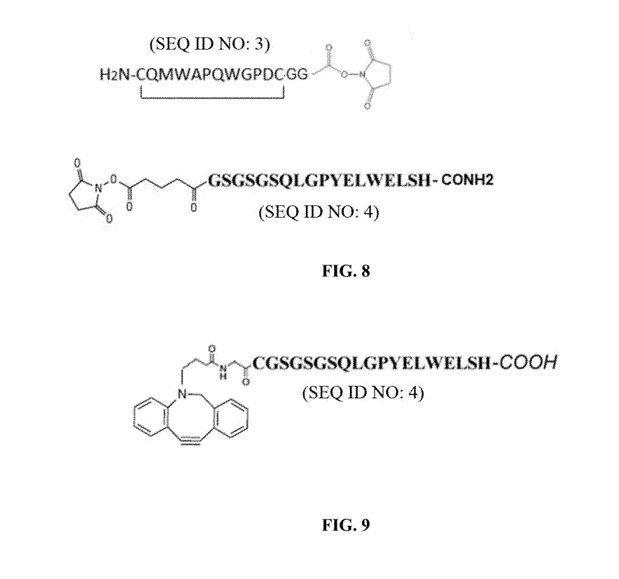

The cell surface anchoring molecule in the antibody binding molecule-optional linker-cell surface anchoring molecule conjugate can also be reactive molecule/functional group that can covalent attach to cell surface molecules such as membrane proteins by chemical reaction once in contact, it has the general structure of antibody binding molecule-optional linker-cell surface reactive moiety conjugate. For example, it can be maleimide containing molecules to react with —SH of cell surface proteins. It can also be activated —COOH ester group such as NHS ester to react with amine group at cell surface to from an amide bind. Examples of Herceptin mimotope peptide conjugate containing an NHS ester are shown in FIG. 8.

Cell surface anchoring can also be done by modification of cell surface glycoprotein through oligosaccharide biosynthesis (e.g. using metabolic cell-labeling to introduce azide group on cell surface and then conjugate antigen with it using click chemistry such as those described in Nature Chemical Biology volume 13:415-424 and Nature. 2004 Aug 19; 430(7002):873-877.

For example, the trigger-activatable Ac3ManAz derivatives such as DCL-AAM described in Nature Chemical Biology volume 13: 415-424 is given to the subject in need, therefore their cancer cell surface will have a —N3 group, next DBCO labeled mimotope peptide (example see FIG. 9) is given to the subject either as IV injection or intratumoral injection, the DBCO will react with —N3 and the cell surface is labeled with mimotope. Other click chemistry compatible alkyne can also be used to label the mimotope.

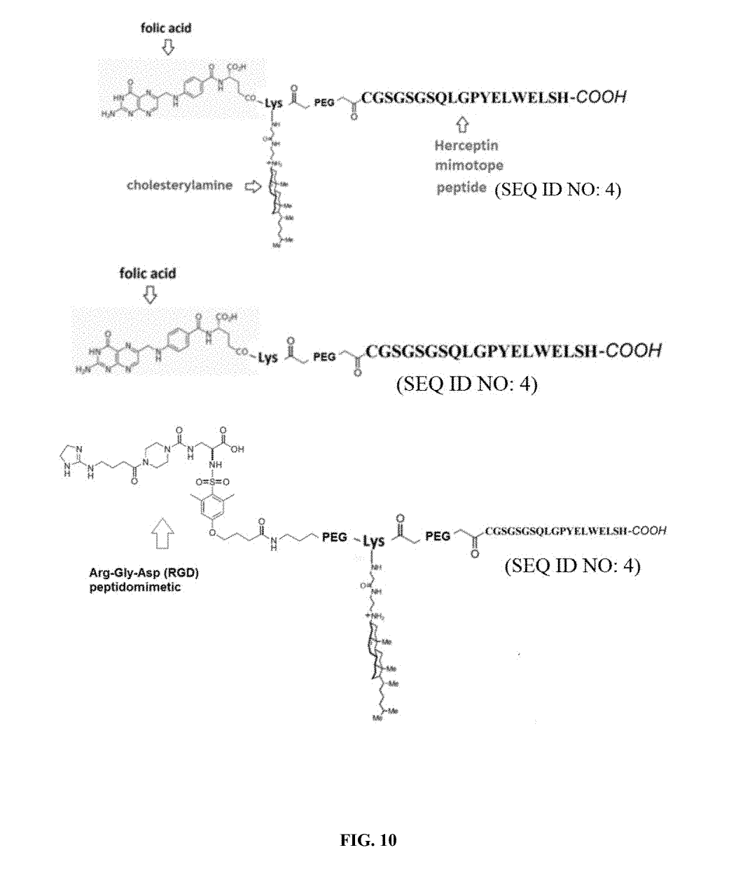

The conjugate can further comprise a cancer cell binding domain to increase its targeting to cancer cell, which will also allow intravenous (iv) or IM or SC injection instated of intratumoral injection. Small molecule ligand for cancer such as folic acid and RGD peptide/peptidomimetic can be used for cancer targeting (e.g. those described in Curr Med Chem. 2014; 21(14):1618-30; Current pharmaceutical design 16(9): 1040-54 and Journal of Amino Acids, Volume 2012 (2012), Article ID 967347). Folic acid or RGD peptide can be incorporated into the conjugate to increase cancer targeting, multievent strategy and aptamer or antibody or its fragment or antibody mimetic type affinity ligand can also be used. Therefore the antibody binding molecule-optional linker-cell surface anchoring molecule conjugate has the structure of antibody binding molecule-optional linker-affinity ligand for cancer cell surface molecule conjugate, with optional cell membrane inserting lipid like molecule as shown in FIG. 10. It can also be simply a Fc fused affinity ligand for cancer cell surface molecule, such as Fc-Anticalin against cell surface molecule, FcMBL (Fc fused mannose binding lectin) that can bind with cancer cell. The affinity ligand can be not specific to cancer cell surface marker if it is injected intratumorally as local injection will generate enough local binding. It can be the ligand for none cancer specific cell surface molecule such as EpCAM. Preferably the Fc can be either isotype or engineered to have high complement activation activity and Fc receptor binding activity to boost antigen presenting. The result Fc anchored to cancer cell surface will induce ADCC effect and improve cancer cell antigen presenting. Preferably the antibody or antibody mimetic or conjugate used in the current invention has long cell surface half life. Examples of antibody mimetic include Anticalin, nanobody/single domain Ab, Affibody, Affimer or the like. Examples of them can be found at Antibody_mimetic in en.wikipedia.org/wiki/Antibody_mimetic.

Administering the resulting conjugate to the patient can be used to treat cancer. Small protein ligand for cancer can also be used. Several examples of the conjugate are: mimotope-linker (optional)-EGF, mimotope-linker (optional)-VEGF, mimotope-linker(optional)-TGF-α, mimotope-GnRH. Preferably affinity ligand that can bind with EGFR or VEGFR without activating them, e.g. EGFR or VEGF antagonist, is used to prepare the conjugate. For example, Decorin, VEGF165b, VEGF antagonist in PCT/CA2010/000275 can be used to prepare the conjugate instead of using native VEGF that can activate VEGFR for angiogenesis. The conjugate of other antigen with peptide/protein/small molecules (e.g. folic acid, VEGF or their derivatives/mimics such as VEGF165b and those disclosed previously) that can bind with cancer cells can be used to treat cancer. Examples of them include folic acid-optional linker-mimotope, VEGF165b-optional linker-mimotope, VEGF-optional linker-mimotope, folic acid-optional linker-mimotope, VEGF165b-optional linker-antigen, VEGF-optional linker-antigen. Examples of conjugates are shown in the FIG. 10.

The said antibody binding molecule-optional linker-cell surface anchoring molecule conjugate is to introduce Fc onto cancer cell surface upon Intratumoral injection, which will kill the cancer cell and enhance tumor antigen presenting by ADCC, complement activation and Fc mediated phagocytosis to enhance APC. An alternative method and agent to attach antibody Fc domain to cancer cell surface is to use Fc (or its fragment)-optional linker-cell surface anchoring molecule conjugate instead. The cell surface anchoring molecule can be the same as those described above. Once being injected intratumorally, preferably in combination with a vaccine adjuvant type agent as described above, it will turn the tumor into an in situ cancer vaccine. Preferably the

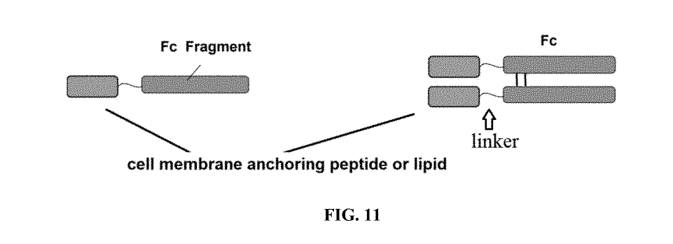

Fc can be either the Fc isotype having (or engineered/mutated to have) high complement activation activity and Fc receptor binding activity to boost antigen presenting. Examples of Fc-optional linker-cell surface anchoring molecule conjugate include Fc-3β-cholesterylamine conjugate, Fc-lipid conjugate, Fc-cell membrane anchoring peptide conjugate and Fc-affinity ligand to cell surface molecule conjugate. They are essentially the conjugate by replacing the antibody binding molecule of above described antibody binding molecule-optional linker-cell surface anchoring molecule conjugate with Fc or its fragment. Example is shown in FIG. 11.

Another agent that can be injected to the tumor to treat cancer is sialidase or sialidase conjugated with cholesterylamine or lipid type molecule. It can increase the cytotoxicity of NK cell and antibody mediated complement activation against tumor cells and activate immune cells. The sialidase can be either bacterial sialidase or viral sialidase or animal sialidase or human sialidase in therapeutical effective amount (e.g. 0.1˜10 mg per injection). It can be either in monomer or oligomer or polymer (e.g. conjugated to a soluble polymer backbone) or coated on nano/micro particles. Preferably it is injected together with the cancer cell inactivating agent into the tumor at therapeutical effective amount. It can be co-formulated with the vaccine adjuvant type agent.

The cancer cell inactivating agent is not limited to antigen-optional linker-cell membrane anchoring molecule conjugate. It can be any agent that can lyse the cancer cell when intratumoural injected. For example, they can be acid or base (e.g. 0.1˜1M pH=2 lactic acid buffer, 0.1˜1 M pH=10 NaCO3 buffer), organic solvent (e.g. 75% ethanol, DMF, DMSO, acetone), perforin, C3b, C5b, membrane attack complex and cell inactivating detergent/surfactant. They can be either in the form of active drug, prodrug, liposome, micelle, conjugated to polymer drug carrier (e.g. dextran) or encapsulated in biodegradable micro particle/nano particle. The preferred amount and concentration should be enough to lyse significant amount of the cancer cells (e.g. >5% of the cancer cells in the tumor being injected). Cell inactivating peptide and antibiotics such as polymyxin are also detergent like compound, which can be used in the current invention. Examples of the detergent that can be used include anionic detergents, cationic detergents, non-ionic detergents and zwitterionic detergents such as alkylbenzenesulfate, alkylbenzenesulfonates, bile acids, deoxycholic acid, quaternary ammonium type detergents, tween, triton, CHAPS, SLS, SDS,SLES, DOC, NP-40, Cetrimonium bromide (CTAB), cetylpyridinium chloride (CPC),Benzalkonium chloride (BAC),benzethonium chloride (BZT),dimethyldioctadecylammonium chloride and dioctadecyldimethylammonium bromide (DODAB) as long as they can effectively lyse the tumor cell in vivo. For example, they can be used as injection at the concentration between 0.1˜100 mg/mL.

The current invention also discloses novel compositions/formulations to treat tumor cell and cancer. The formulation comprises one or more said cancer cell inactivating agent and/or agent can enhance cancer cell antigen presenting (antigen presenting booster) and immune activity enhancing agent in a pharmaceutical acceptable carrier. It can be injectable solution or solid dosage form such as lyophilized formulation that can be reconstituted to injectable solution. The formulation contains cancer cell inactivating agent/antigen presenting booster and immune activity enhancing agent as well as pharmaceutical acceptable excipients suitable for injection. They can be in form of active drug, prodrug, liposome, micelle, emulsion, gel formulation, implant, thermal phase changing formulation, insoluble precipitate (e.g. in complex with condensing agent), conjugated to polymer drug carrier (e.g. dextran) or coated on or encapsulated in biodegradable micro particle/nano particle. Suitable size of the particle is between 10 nm˜100 um.

Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into a patient in need thereof, and are, in particular, focused on intravenous delivery.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents.

In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms.

Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES. In certain embodiments, an appropriate buffer system (e.g., sodium phosphate, sodium acetate, sodium citrate, sodium borate or boric acid) is added to the composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 2 to about pH 11. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7.

In making pharmaceutical compositions that include cell surface anchoring conjugates described herein, the active ingredient is usually diluted by an excipient or carrier and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of films, gels, powders, suspensions, emulsions, solutions, containing, for example, up to 10% by weight of the active compounds, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: wetting agents; emulsifying and suspending agents; and preserving agents such as methyl- and propylhydroxy-benzoates. Liquid solution as used herein refers to solutions, suspensions, emulsions, drops, ointments, liquid wash, sprays, liposomes which are well known in the art. In some embodiments, the liquid solution contains an aqueous pH buffer agent which resists changes in pH when small quantities of acid or base are added.

Alternatively, exemplary formulations may comprise: a) cell surface anchoring conjugate and immune function enhancing agents as described herein; b) pharmaceutically acceptable carrier; and c) hydrophilic polymer as matrix network, wherein said compositions are formulated as viscous liquids, i.e., viscosities from several hundred to several thousand cps, gels or ointments. In these embodiments, the cell surface anchoring antigen conjugates is dispersed or dissolved in an appropriate pharmaceutically acceptable carrier.

In certain embodiments, the cell surface anchoring conjugates or a composition comprising the same, is lyophilized prior to, during, or after, formulation. In certain embodiments, the cell surface anchoring conjugates, or a composition comprising the same, is lyophilized in a pharmaceutical formulation comprising a bulking agent, a lyoprotectant, or a mixture thereof. In certain embodiments, the lyoprotectant is sucrose. In certain embodiments, the bulking agent is mannitol. In certain embodiments, the cell surface anchoring conjugates, or a composition comprising the same, is lyophilized in a pharmaceutical formulation comprising mannitol and sucrose. Exemplary pharmaceutical formulations may comprise about 1-20% mannitol and about 1-20% sucrose. The pharmaceutical formulations may further comprise one or more buffers, including but not limited to, phosphate buffers. Accordingly, also provided herein is a lyophilized composition comprising a drug conjugate, nanoparticle or composition comprising the same as described herein.

Suitable dosages can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.0001 mg to about 10 mg. In other illustrative aspects, effective doses ranges from about 0.01 μg to about 1000 mg per dose, 1 μg to about 100 mg per dose, or from about 100 μg to about 50 mg per dose, or from about 500 mg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In any of the various embodiments described herein, effective doses ranges from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 μg to about 10 μg per dose. In other illustrative embodiments, effective doses can be about 1 μg, about 10 μg, about 25 μg, about 50 μg, about 75 μg, about 100 μg, about 125 μg, about 150 μpg, about 200 μg, about 250 μg, about 275 μg, about 300 μg, about 350 μg, about 400 μg, about 450 μg, about 500 μg, about 550 μg, about 575 μg, about 600 μg, about 625 μg, about 650 μg, about 675 μg, about 700 μg, about 800 μg, about 900 μg, 1.0 mg, about 1.5 mg, about 2.0 mg, about 10 mg, about 100 mg, or about 100 mg to about 30 grams. In certain embodiments, the dose is from about 0.01 mL to about 10 mL.

In certain embodiments, the dose is administered to the subject in need thereof on daily basis as an injection. In other embodiments, the dose is given to the object once every 2-3 days as injection. In other illustrative embodiments, the dose is administered to the subject in need thereof once each week as an injection. In other embodiments, the dose is administered to the subject in need thereof once every two weeks as an injection. In other embodiments, the dose is administered to the subject in need thereof once every month as an injection. The treatment can be continued until the desired therapeutical effect is reached.

The cancer cell inactivating agent can be said antibody binding molecule-optional linker-cell surface anchoring molecule conjugate of the current invention or native antigen-optional linker-cell surface anchoring molecule conjugate in the prior applications (e.g. U.S. patent application Ser. No. 15/945,741 from the current inventor) or their combinations.

In some embodiments, the formulations contain 1˜100 mg/mL cancer cell inactivating agent/antigen presenting booster (e. g. Her2 —cholesterylamine or Her2 epitope for Herceptin-cholesterylamine or Herceptin mimotope-cholesterylamine conjugate or their mixture at 1:1 molar ratio), 0.1˜50 mg/mL TLR7/8 Ligands (e.g. imiquimod or gardiquimod or resiquimod), 0.1˜50 mg/mL TLR3/RLR Ligands (e.g. dsRNA such as poly IC or polyICLC), 0.1˜50 mg/mL TLR9 Ligands (e.g. CpG ODNs such as ODN 1826 or ODN 2216) and optional 0.1˜50 mg/mL neuraminidase (Sialidase) from Vibrio cholera and optional 0.1˜50 mg/mL Herceptin in 1×PBS, then being lyophilized to give the final formulation. In one example, the formulations contain 30 mg/mL cancer cell inactivating agent/antigen presenting booster (e. g. Herceptin mimotope—cholesterylamine conjugate or Herceptin mimotope-cell membrane anchoring peptide conjugate), 5 mg/mL imiquimod, 5 mg/mL poly IC, 5 mg/mL classe A CpG ODN 2216, optional 100 mg/mL Herceptin, and 5 mg/mL neuraminidase (Sialidase) from Vibrio cholera in 1×PBS and 5% sucrose. It can be injected to the tumor at 100 uL˜300 uL/cm3 tumor size after being reconstituted with water. In another example, the formulations contain 100 mg/mL cancer cell inactivating agent/antigen presenting booster (e. g. Herceptin mimotope—cholesterylamine conjugate or Herceptin mimotope-cell membrane anchoring peptide conjugate), 2 mg/mL imiquimod, 2 mg/mL poly IC, 2 mg/mL class A CpG ODN 2216 or class B CpG ODN, 10 mg/ml Herceptin and 2 mg/mL neuraminidase (Sialidase)-lipid conjugate in 1×PBS and 15% mineral oil to form an emulsion.

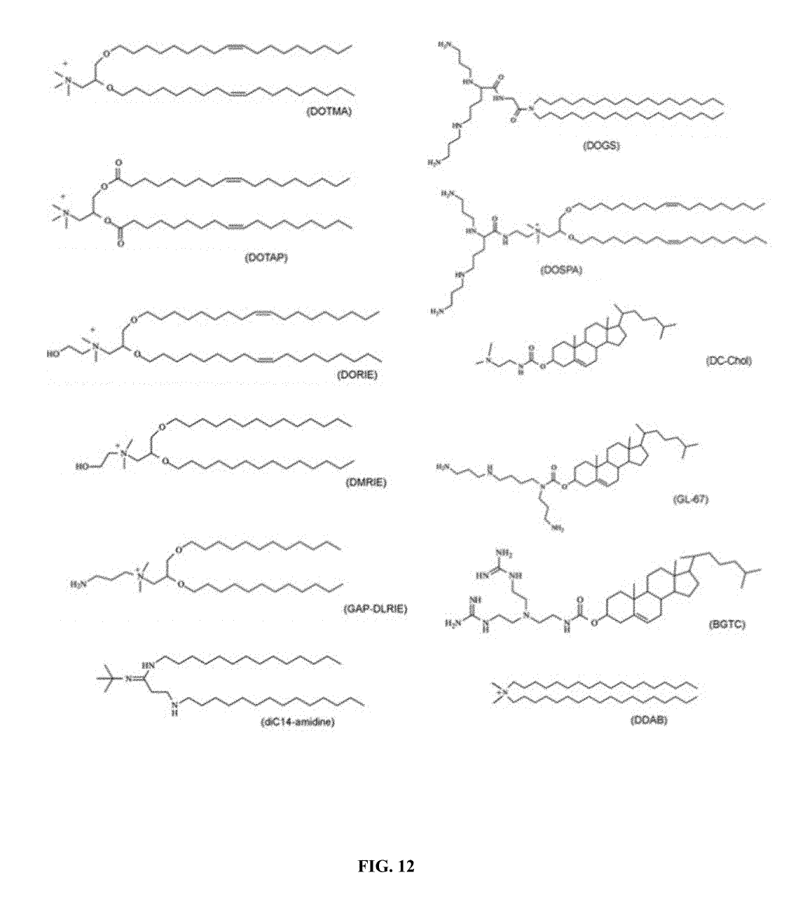

The drugs in the above embodiments are in active form, one or more or all of them can also be in the form of prodrug, liposome, micelle, insoluble precipitate (e.g. in complex with condensing agent), conjugated to polymer drug carrier (e.g. dextran) or coated/adsorbed on or encapsulated in biodegradable micro particle/nano particle as previously described. For example, compounds having one or more amine groups that can precipitate poly IC and CpG ODN therefore generate water insoluble precipitates that can be used as sustained release drug form for the current invention. Examples of said co-precipitation compound include α-polylysine, ε-polylysine, spermine, polymyxin, gentamycin, nisin, DC-Cholesterol, cholesterylamine, tertiary/quaternary ammonium type detergents (e.g. Cetrimonium salt, cetylpyridinium salt,Benzalkonium chloride,benzethonium chloride,dimethyldioctadecylammonium chloride and dioctadecyldimethylammonium salt) or their base form. Imiquimod or gardiquimod or resiquimod can also form precipitation with poly IC or CpG ODN or other anionic polymer or anionic lipid or anionic surfactant, which can be used in the current invention. Surfactant can be added to the precipitates to from stable suspension. Cationic lipid can also be used as co-precipitation anionic compound, such as 3β-[N-(N′,N′-Dimethylaminoethane) Carbamoyl] Cholesterol, MVL5, DOTMA, ETHYL PC, DDAB, DC-CHOLESTEROL from Avantilipids. Examples of cationic lipids that can be used are shown in FIG. 12. Other co-precipitation compound that can be used include lipophilic drug having positively charged group such as Phentermine type drug, Dyclonine, Decamethonium, Meclofenoxate, Cyprodenate, Propantheline bromide, Diphenhydramine, Orphenadrine, Pheniramine, Berberine, positively charged Tricyclic antidepressant such as Amitriptyline, Butriptyline, Clomipramine, Desipramine, Dibenzepin, Dosulepin, Doxepin, Imipramine, Iprindole, Lofepramine, Maprotiline, Norclomipramine, Northiaden, Nortriptyline, Opipramol, Protriptyline, Tianeptine and Trimipramine; positively charged lipophilic anesthetics such as procaine, methocaine, lidocaine, prilocaine, bupivacaine, levobupivacaine, ropivacaine, mepivacaine and dibucaine. They can be mixed with negatively charged poly IC or CpG ODN to form water insoluble complex (precipitation in water) to be used as intratumoral injection. The preparation protocol can be adopted from those described in publications such as patent application PCT/US2003/025415. The formed complex can also be encapsulated in biodegradable micro particle/nano particle and then being injected intratumorally to treat cancer.

Encapsulation of poly IC or CpG ODN in biodegradable micro or nano sphere can be performed by the addition of amine containing compounds described above. For example, PLGA-hybrid nanospheres encapsulating poly IC or CpG ODN is prepared using a double emulsion-solvent evaporation method. Briefly 1 ml poly IC or CpG ODN in Tris/EDTA buffer is emulsified in a PLGA solution (5% w/v in methylene chloride, MW =66,000 Da; Birmingham Polymers, Birmingham, Ala., USA) with DC-Cholesterol or cetyldimethylamine or gardiquimod solution (5% w/v in methylene chloride) using a sonicator for 5 min. A water-in-oil solution is emulsified in 25 ml of 4% (w/v) aqueous polyvinyl alcohol (PVA, MW =30,000-70,000 Da; Sigma, St. Louis, Mo.) solution using a sonicator for 5 min. The emulsion is stirred for 72 h at room temperature to remove methylene chloride. PLGA nanospheres is recovered by ultracentrifugation (20,000g for 20 min at 4° C.). The PLGA nanosphere pellet is washed five times in distilled water to remove PVA and was then re-suspended by vortexing and lyophilizing for 48 h to obtain a dry powder. When additional imiquimod (e.g.1% w/v in methylene chloride) is added to the poly IC or CpG ODN solution, the resulting nanosphere will also encapsulate imiquimod. The prepared nanosphere can be used as vaccine adjuvant for the current invention.

In another example, the nanosphere encapsulating poly IC and imiquimod is prepared using a double emulsion water/oil/water system. Briefly, the PLGA is prepared at 10% wt/vol in CH2Cl2, which also contain 3% imiquimod and poly IC is prepared at 50 mg/mL in PBS. Emulsification via sonication is performed using a homogenizer and then a sonicator. The primary emulsion is carried out in a thick walled glass pressure tube with an aqueous to organic phase ratio of 1:5. Following a homogenization step, Emprove PVA 4-88 aqueous solution is added to the PLGA organic solution (at a volume ratio of 3:1 PVA to organic phase), vortex mixed, and emulsified by sonication. The resultant double emulsion is then transferred into a beaker under stirring containing 70 mM phosphate buffer pH 8.0 at a volume ratio of 1 part double emulsion to 7.5 parts buffer. The organic solvent (CH₂Cl₂) is allowed to evaporate for 2 h under stirring, and the nanoparticles are recovered via centrifugation at 75,600 rcf with two wash steps. PBS is used for the wash solutions and the final resuspension media. The washed suspension is stored at −20 ° C. Other examples of preparing TLR agonist containing particle or precipitations can be found in the disclosure of U.S. patent application Ser. No. 15/945,741.

Besides TLR agonist, other molecules that can activate/boost the function of immune system and immune cell such as APC, B cell and T cells can also be incorporated into the intratumoral injection formulation. Suitable immune function activating/boosting molecule can be selected from granulocyte macrophage colony-stimulating factor (e.g. sargramostim or molgramostim), immunostimulatory monoclonal antibody (e.g. Anti-KIR antibody such as Lirilumab, antibody for CD137 such as Urelumab or Utomilumab), heparan sulfate (HS) mimetics such as PG545 (pixatimod, pINN) , FMS-like tyrosine kinase 3 ligand (FLT3L), other pattern recognition receptor agonists besides poly IC, CpG and imiquimod, T-cell-tropic chemokines such as CCL2, CCL1, CCL22 and CCL17, B-cell chemoattractant such as CXCL13, Interferon gamma, type I IFN (e.g. IFN-a, IFN-beta), tumor necrosis factor (TNF)-beta, TNF-alpha, IL-1, Interleukin-2 (IL-2 such as aldesleukin, teceleukin or bioleukin), interleukin-10 (IL-10), IL-12, IL-6, IL-24, IL-2, IL-18, IL-4, IL-5, IL-6, IL-9 and IL-13 or their derivatives such as PEGylated derivative, CD1d ligand, Vα14/Vβ8.2 T cell receptor ligand, iNKT agonist, α-galactosylceramide (α-GalCer), α-glucosylceramide (α-GlcCer), α-glucuronylceramide, α-galacturonylceramide, Isoglobotriosylceramide (iGb3), HS44 , interleukin 12, antibody against OX 40, tumor necrosis factor, interferon gamma (IFNγ), immunomodulatory imide drugs (immune enhancing IMiDs such as thalidomide, lenalidomide and pomalidomide), Treg inhibitory agent such as inhibitory antibody against Treg (such as antibody against CD4, CD25, FOXP3 and TGF-β or its receptor) or their combinations. CD25 is more abundant in Treg, targeting CD25 provide inhibitory effect to Treg selectively over other cytotoxic T cells. They can be added to the formulation described above at therapeutically effective amount, to be used as an intratumoral injection.

In another example, the formulation is a solution containing 20˜200 mg/mL Cetuximab mimotope-cholesterylamine conjugate or Cetuximab mimotope-cell membrane anchoring peptide conjugate, 3 mg/mL poly IC or 3 mg CpG ODN 2216 or both, 20 mg/mL biodegradable PLGA nano particles encapsulating 20% imiquimod, optional 5-100 mg/mL Cetuximab and granulocyte-monocyte colony-stimulating factor (10-200 μg/mL). Suitable amount of surfactant can be added to from stable suspension. After the patient receive the intratumoral injection with the above formulation at 0.5 mL/cm3 tumor volume, the patient is intravenously injected with Cetuximab immediately 3˜10 mg/kg once and Ipilimumab 3˜10 mg/kg every 3 weeks for 4 doses, or Atezolizumab 1200 mg IV q3wk until disease progression. Cetuximab 3˜10 mg/kg can also be is intravenously injected before the intratumoral injection of the above formulation. 20˜200 mg/mL L-rhamnose-cholesterylamine conjugate can also be added to the formulation.

Herceptin mimotope-lipid conjugate with optionally 100 mg/mL Herceptin, 10 mg/mL imiquimod, 2 mg/mL poly IC, 2 mg/mL CpG ODN 2216, 1×10⁴-1×10⁵ U/mL of IFN-α, 1-10 MIU/mL IL-2, L-Arginine, L-cysteine and L-tryptophan at 20˜100 mg/mL, poly aspirin 20 mg/mL, glutathione or SOD 5 mg/mL, N-hydroxy-L-Arginine 10 mg/mL, tadalafil 3 mg/mL, axitinib 10 mg/mL, Nitro-aspirin 5 mg/mL, all-trans retinoic acid 5 mg/mL, 5 mg/mL α-GalCer, Gemcitabine 10 mg/mL, cucurbitacin 10 mg/mL. Suitable amount of carbomer is added to the solution to reach a viscosity of 1,000,000 cps. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression. Herceptin 5-10 mg/kg can also be intravenously injected before or after the intratumoral injection of the above formulation.

In another example, the formulation is a solution containing 100˜200 mg/mL PLGA nano particles encapsulating 20% Herceptin mimotope-lipid conjugate, 2 mg/mL antibody against OX40, 2 mg/mL poly IC, 2 mg/mL CpG ODN 2216, 5 mg imiquimod, 0.5-2 mg/mL α-GalCer, 25×10⁴ U/mL of IFN-α, 5 MIU/mL IL-2. After the patient receive the intratumoral injection with the above formulation at 0.3 mL/cm3 tumor volume, the patient is intravenously injected with Ipilimumab 3˜10 mg/kg every 3 weeks for 4 doses, or Atezolizumab 1200 mg IV q3wk until disease progression. Herceptin 5-10 mg/kg is intravenously injected right before or right after the intratumoral injection of the above formulation.

In another example, the formulation is a solution containing 100˜200 mg/mL PLGA nano particles encapsulating 20% Herceptin mimotope-folate conjugate, 20˜200 mg/mL alpha-gal-cholesterylamine conjugate, 2 mg/mL poly IC, 2 mg/mL CpG ODN 2216, 5 mg 3M-052, 5 MIU/mL IL-2. After the patient receive the intratumoral injection with the above formulation at 0.6 mL/cm3 tumor volume, the patient is intravenously injected with Ipilimumab 3˜10 mg/kg every 3 weeks for 4 doses, or Atezolizumab 1200 mg IV q3wk until disease progression. Herceptin 5-10 mg/kg is intravenously injected right before or right after the intratumoral injection of the above formulation.

In another example, the formulation is a solution containing 100˜200 mg/mL Fc-lipid conjugate or FcMBL, 10 mg/mL imiquimod, 2 mg/mL poly IC, 2 mg/mL CpG ODN 2216, 50 μg/mL granulocyte-monocyte colony-stimulating factor, 1×10⁴-1×10⁵ U/mL of IFN-α, 1-10 MIU/mL IL-2. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression.

In another example, the formulation is a solution containing 100˜200 mg/mL Herceptin mimotope NHS ester, 10 mg/mL imiquimod, 2 mg/mL poly IC, 2 mg/mL CpG ODN 2216. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression. Herceptin 5-10 mg/kg can also be intravenously injected before or after the intratumoral injection of the above formulation.

Instead of the antigen containing conjugate described above, another type of cancer cell inactivating agent can be used for the current invention is therapeutic antibody including monoclonal antibody, bi-specific antibody and antibody-drug conjugate as well as cytotoxic T cell used to treat cancer. Examples of therapeutic antibody including antibody-drug conjugate include Herceptin, Rituximab, Bexxar, Cetuximab, Bevacizumab, Panitumumab, Pertuzumab, Kadcyla and Catumaxomab, antibody against tumor surface antigen such as GalNAc—O-Ser/Thr (Tn Antigen), Gal 1-3GalNAc—O-Ser/Thr (Core 1 antigen), STF Antigen and etc. The antigen need not to be highly tumor specific because the antibody can be injected into the tumor to reach high local concentration to be effective. For example, antibody against Epithelial cell adhesion molecule (EpCAM) antigen can be used for epithelia and epithelial-derived tumor cells although it also binds with other normal epithelial cells. Preferably the antigen for the antibody to be used is highly abundant on cancer cell surface (but not need to be cancer cell specific), so the antibody will bind with cancer cell surface extensively. Example of highly abundant cell surface protein include CD98, sushi repeat-containing protein, chaperone proteins including GRP78, GRP75,

HSP70, HSP60, HSP54, HSP27, and protein disulfide isomerase. It can also be antibody against cell surface carbohydrate such as mannose or sialic acid or lipid. These antibody or antibody-drug conjugate can be injected into solid tumor to lyse the tumor cell and/or improve the antigen presenting, therefore release neo antigen to promote immune response. Preferably they are injected into the tumor together with immune activity enhancing agent such as vaccine adjuvant and optionally with sialidase. Preferably the target tumor need to have expression of the antigen specific for the antibody, e.g. the tumor needs to be HER2+ for treatment using Herceptin and the tumor needs to be EGFR-expressing for treatment using Cetuximab. The formulation suitable for the current invention includes one or more antibody type drug and immune activity enhancing agent and optional sialidase at therapeutically effective amount.

In one example, the formulation contains 2˜100 mg/mL Herceptin, 2 mg/mL imiquimod, 2 mg/mL poly IC, 5 mg/mL α-GalCer and 2 mg/mL neuraminidase (Sialidase, human) in 1×PBS. It can be injected into the Her2 positive tumor at 100˜500 uL/cm3 tumor volume to treat cancer every 10 days for total 3 times. Check point inhibitor can be given to the patient at the same time and later.

In one example, the formulation contains 2˜100 mg/mL Herceptin, L-Arginine, L-cysteine and L-tryptophan at 2˜100 mg/mL, Celecoxib 20 mg/mL, curcumin or BHT 20 mg/mL, cyclophosphamide 10 mg/mL, 2 mg/mL imiquimod, 2 mg/mL poly IC, 5 mg/mL α-GalCer and 2 mg/mL neuraminidase (Sialidase, human) in 1×PBS. Suitable amount of hyaluronic Acid is added to the solution to reach a viscosity of 5,000,000 cps. It can be injected into the Her2 positive tumor at 100˜500 uL/cm3 tumor volume to treat cancer every 10 days for total 3 times. Check point inhibitor can be given to the patient at the same time and later.

In one example, the formulation contains 20˜100 mg/mL humanized antibody against EpCAM, 2 mg/mL imiquimod, 2 mg/mL poly IC, L-Arginine, L-cysteine and L-tryptophan at 20˜100 mg/mL, poly aspirin 20 mg/mL, tadalafil 3 mg/mL, Nitro-aspirin 5 mg/mL, all-trans retinoic acid 5 mg/mL, 5 mg/mL α-GalCer, Gemcitabine 10 mg/mL, cucurbitacin 10 mg/mL, and 2 mg/mL neuraminidase (Sialidase, human) in 1×PBS. It can be injected into the tumor at 100˜500 uL/cm3 tumor volume to treat cancer every 10 days for total 3 times. Check point inhibitor can be given to the patient at the same time and later.

In another example, the formulation contains 50 mg/mL Herceptin with optional 50 mg/mL Cetuximab, 1 mg/mL imiquimod and 2 mg/mL neuraminidase in pharmaceutical acceptable excipient. In another example, the formulation contains 50 mg/mL Trastuzumab emtansine, 20 mg/mL PLGA nanoparticle containing 10% imiquimod and 2 mg/mL poly IC in pharmaceutical acceptable excipient.

In another example, the formulation contains 50 mg/mL Cetuximab, 2-5 mg/mL imiquimod and 2 mg/mL neuraminidase (Vibrio cholera) in PBS. In another example, the formulation contains 50 mg/mL Cetuximab, 20 mg/mL PLGA nanoparticle containing 10% imiquimod, 2 mg/mL antibody against OX40, 2 mg/mL poly IC, 50 μg/mL granulocyte-monocyte colony-stimulating factor, 1×10⁴-1×10⁵ U/mL of IFN-α, 1-10 MIU/mL IL-2 in pharmaceutical acceptable excipient. These Cetuximab containing formulations can be injected into EGFR-expressing tumor at 100˜500 uL/cm3 tumor volume to treat cancer every 10 days for total 3 times. Check point inhibitor can be given to the patient at the same time and later.

In another example, the formulation is a suspension containing 50 mg/mL Herceptin and 5 mg/mL imiquimod in pharmaceutical acceptable excipient. The antibody type drug can be mixed with other ingredient right before injection, therefore allow the user to use the commercially available antibody type drug, e.g. the user can use the formulation solution containing vaccine adjuvant with optional sialidase as diluents to reconstitute the lyophilized antibody drug; or use the commercially available antibody drug solution as diluents to reconstitute the lyophilized formulation containing vaccine adjuvant and sialidase.

Similarly, chemotherapy drug can also be used as cancer cell inactivating agent in the current invention. Example of these drugs include alkylating agents (such as Cyclophosphamide, Uramustine, Carmustine and Usulfan), Antimetabolites (such as methotrexate and fluorouracil),

Anti-microtubule agents (such as paclitaxel, vindesine, and vinflunine), Topoisomerase inhibitors(such as irinotecan and topotecan) and Cytotoxic antibiotics (such as anthracyclines, bleomycins, mitomycin C, mitoxantrone, and actinomycin).

The current invention discloses antibody binding molecule-optional linker-cell surface anchoring molecule conjugate and its use to treat cancer. The main propose of the antibody binding molecule moiety is to increase antigen presenting for tumor associated antigen, which is mainly from the Fc moiety of the antibody introduced by the said antibody binding molecule. Similarly, besides Fc moiety introduced by antibody binding molecule, other molecule (or can be called as moiety) that can increase cancer cell antigen presenting can also be used to build the conjugate instead of the antibody binding molecule. The general structure of the conjugate is antigen presenting enhancing molecule-optional linker-cell surface anchoring molecule conjugate and its use can be similar to the use of the above antibody binding molecule-optional linker-cell surface anchoring molecule conjugate, e.g. by replacing the antibody binding molecule-optional linker-cell surface anchoring molecule conjugate in the above examples and embodiments with antigen presenting enhancing molecule-optional linker-cell surface anchoring molecule conjugate. For example, antigen presenting enhancing molecule can be affinity ligand (e.g. antibody, antibody fragment, antibody mimetics, aptamer) for antigen presenting cells (e.g. their cell surface marker). Examples of antigen presenting cells include

Dendritic cells, Macrophages, B cells, T cells and NK cells. For example, antigen presenting enhancing molecule can be antibody against DC cell surface marker, e.g. antibody (or its fragment) against CD11C or affinity ligand for macrophage such as Fab against CD14. Other examples include affinity ligand for pattern recognition receptors (PRRs) such as the toll-like receptors (TLRs), which are widely expressed on APC surface.

One of the main type of antigen presenting enhancing molecule is the molecule that can enhance endocytosis. Therefore, the conjugate that can be used in the current invention has the structure of endocytosis enhancing molecule-optional linker-cell surface anchoring molecule conjugate. Endocytosis pathways can be subdivided into four categories: namely, receptor-mediated endocytosis (also known as clathrin-mediated endocytosis), caveolae, macropinocytosis, and phagocytosis. In some embodiments, the conjugate that can be used in the current invention has the structure of phagocytosis enhancing molecule-optional linker-cell surface anchoring molecule conjugate.

Phagocytosis in mammalian immune cells is activated by attachment to pathogen-associated molecular patterns (PAMPS). Opsonins such as C3b and antibodies can also act as attachment sites and aid phagocytosis of pathogens. Cell presentation of a variety of intracellular molecules on the cell surface, such as calreticulin, phosphatidylserine, annexin Al, oxidised LDL and altered glycans can enhance apoptosis by efferocytosis. These molecules are recognized by receptors on the cell surface of the macrophage such as the phosphatidylserine receptor or by soluble (free-floating) receptors such as thrombospondin 1, GAS6, and MFGE8, which themselves then bind to other receptors on the macrophage such as CD36 and alpha-v beta-3 integrin. Therefore these molecules can also be used as phagocytosis enhancing molecule to construct the conjugate.

Examples of the phagocytosis enhancing molecule include pathogen-associated molecular patterns (PAMPS) molecule, pattern recognition receptors (PRRs) including secreted Pattern recognition receptors (PRRs) such as Pentraxins, collectins, ficolins, sCD14, MFG-E8, natural IgM and C1q, complement system proteins such as C1q, C3b, C4 and C3-convertase, antibody and its fragment such as Fc and their mimetics. They can be conjugated to the cell surface anchoring molecule to form antigen presenting enhancing molecule-optional linker-cell surface anchoring molecule conjugate, which can be injected intratumorally in combination with said immune activity enhancing agent to form in situ vaccine against tumor. If they themselves have affinity to cells or can be deposited around the cell after injection, the conjugation may not be required, examples include C1q, C3b, C4, C3-convertase and secreted Pattern recognition receptors (PRRs), which can be injected into the tumor directly without conjugation.

In some embodiments, the conjugate is toll-like receptors (TLRs) ligand-optional linker-cell surface anchoring molecule conjugate. Examples of TLR ligand include bacterial carbohydrates (such as lipopolysaccharide or LPS, mannose), nucleic acids (such as bacterial or viral DNA or RNA), bacterial peptides (flagellin, microtubule elongation factors), peptidoglycans and lipoteichoic acids (from Gram-positive bacteria), N-formylmethionine, lipoproteins, fungal glucans and chitin, synthetic TLR ligands such as imidazoquinoline, CpG ODNs and poly IC.

Examples of these conjugate include CpG ODN-fatty acid conjugate, CpG ODN-cholesterylamine conjugate, CpG ODN-cell membrane anchoring peptide conjugate, CpG ODN-folate conjugate, Poly IC-fatty acid conjugate, Poly IC-cholesterylamine conjugate, Poly IC-cell membrane anchoring peptide conjugate, Poly IC-folate conjugate, Imiquimod-fatty acid conjugate, Imiquimod-cholesterylamine conjugate, Imiquimod-cell membrane anchoring peptide conjugate, Imiquimod-folate conjugate, C1q-fatty acid conjugate, C1q-cholesterylamine conjugate, C1q-cell membrane anchoring peptide conjugate, C1q-folate conjugate. Other cell surface anchoring molecule and other endocytosis enhancing molecule can also be used to make the conjugate. The resulting conjugate can be either used alone or in combination with other antibody binding molecule-optional linker-cell surface anchoring molecule conjugate as intratumoral injection. The toll-like receptors (TLRs) ligand-optional linker-cell surface anchoring molecule conjugate can also be used as regular TLR agonist described previously (e.g. similar to the use of imidazoquinoline, CpG ODNs and poly IC).

In one example, the formulation contains 2˜50 mg/mL C1q-lipid conjugate or C3b or C3-convertase, 2 mg/mL imiquimod, 2 mg/mL poly IC, 5 mg/mL α-GalCer and 2 mg/mL neuraminidase (Sialidase, human) in 1×PBS. It can be injected into the tumor at 100˜500 uL/cm3 tumor volume to treat cancer every 10 days for total 3 times. Check point inhibitor can be given to the patient at the same time and later.

In one example, the formulation contains 10˜100 mg/mL CpG ODN-cholesterylamine conjugate or poly IC-cholesterylamine conjugate, 5 mg/mL α-GalCer and 2 mg/mL neuraminidase (Sialidase, human) in 1×PBS. It can be injected into the tumor at 100˜500 uL/cm3 tumor volume to treat cancer every 10 days for total 3 times. Check point inhibitor can be given to the patient at the same time and later.

In another example, the formulation is a solution containing 100˜200 mg/mL Herceptin mimotope-lipid conjugate, CpG ODN 2216-fatty acid conjugate, 10 mg/mL imiquimod, 2 mg/mL poly IC, 10 mg/mL antibody against CD25, 10 mg/mL antibody against OX40, 50 μg/mL granulocyte-monocyte colony-stimulating factor, 1×10⁴-1×10⁵ U/mL of IFN-α, 1-10 MIU/mL IL-2. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression. Herceptin 5-10 mg/kg can also be intravenously injected before or after the intratumoral injection of the above formulation.

In one example, the formulation contains 10˜100 mg/mL imiquimod-cholesterylamine conjugate or CpG ODN-folate conjugate, 5 mg/mL α-GalCer and 2 mg/mL neuraminidase-lipid conjugate in 1×PBS. It can be injected into the tumor at 100˜500 uL/cm3 tumor volume to treat cancer every 10 days for total 3 times. Check point inhibitor can be given to the patient at the same time and later.

In one example, the formulation contains 10˜100 mg/mL CpG ODN-cell membrane anchoring peptide conjugate or C3 convertase-lipid conjugate, 5 mg/mL α-GalCer in 1×PBS. It can be injected into the tumor at 100˜500 uL/cm3 tumor volume to treat cancer every 10 days for total 3 times. Check point inhibitor can be given to the patient at the same time and later.

Optionally Nonsteroidal Anti-inflammatory Drugs (NSAIDs) can be added to the composition/formulation of the current inventions and those in U.S. patent application Ser. No. 15/945,741 to be injected intratumorally to treat cancer. Suitable amount can be between 0.01˜5% w/w. Examples of NSAIDs can be used include COX-1 and/or COX-2 inhibitors such as aspirin, poly aspirin, Salicylic acid, Salsalate, Ibuprofen, Naproxen, Loxoprofen, Diflunisal, Piroxicam, Meloxicam, Tenoxicam, Droxicam, Lornoxicam, Phenylbutazone, Mefenamic acid, Meclofenamic acid, Flufenamic acid, Celecoxib, Etoricoxib. They can be in form of active drug, prodrug, liposome, emulsion, micelle, insoluble precipitate (e.g. in complex with condensing agent), conjugated to polymer drug carrier (e.g. dextran), coated on or encapsulated in biodegradable micro particle/nano particle (e.g. those made of biodegradable polymer such as PLA, PLGA, PCL, PGA or PHB). Suitable size of the particle can be between 10 nm˜100 um.

Optionally L-Arginine or L-cysteine or L-tryptophan or their combinations can be added to the composition/formulation of the current inventions and those in U.S. patent application Ser. No. 15/945,741to be injected intratumorally to treat cancer. Suitable amount can be between 0.01˜5% w/w. They can be in form of active molecule, prodrug (e.g. their ethyl ester), liposome, emulsion, micelle, insoluble precipitate (e.g. in complex with condensing agent), conjugated to polymer drug carrier (e.g. dextran), coated on or encapsulated in biodegradable micro particle/nano particle (e.g. those made of biodegradable polymer such as PLA, PLGA, PCL, PGA or PHB). Suitable size of the particle can be between 10 nm˜100 um. Enzyme inhibitor that can prevent their depletion can also be used, e.g. indoleamine 2,3-dioxygenase inhibitor that can block the depletion of tryptophan in the tumor.

Optionally free radical scavenger/antioxidant can be added to the composition/formulation of the current inventions and those in U.S. patent application Ser. No. 15/945,741 to be injected intratumorally to treat cancer. Suitable amount can be between 0.01˜5% w/w. They can be in form of active molecule, prodrug, liposome, emulsion, micelle, insoluble precipitate (e.g. in complex with condensing agent), conjugated to polymer drug carrier (e.g. dextran), coated on or encapsulated in biodegradable micro particle/nano particle (e.g. those made of biodegradable polymer such as PLA, PLGA, PCL, PGA or PHB). Suitable size of the particle can be between 10 nm˜100 um. Examples of free radical scavenger/antioxidant include vitamin C, Vitamin E, curcumin, BHT, BHA, tea polyphenol, glutathione and enzymes (e.g., catalase and superoxide dismutase).

Optionally agent that can inactivate Treg and/or inhibit tumor-associated macrophage can be added to the composition/formulation of the current inventions and those in U.S. patent application Ser. No. 15/945,741 to be injected intratumorally to treat cancer. Suitable amount can be between 0.001˜5% w/w. They can be in form of active molecule, prodrug, liposome, emulsion, micelle, insoluble precipitate (e.g. in complex with condensing agent), conjugated to polymer drug carrier (e.g. dextran), coated on or encapsulated in biodegradable micro particle/nano particle (e.g. those made of biodegradable polymer such as PLA, PLGA, PCL, PGA or PHB). Suitable size of the particle can be between 10 nm˜100 um. Examples of agent that can inactivate Treg include PI3K-Akt pathway inhibitors such as P13K inhibitor Wortmannin (WM) and the Akt inhibitor triciribine (TCN), antibody against neuropilin-1, cytotoxic antibody against Foxp3+, antibody against activator of nuclear receptor kappa-B ligand (RANKL), antibody against CD73, antibody against CD39, chemotherapeutic agents that can limit Treg-cell function and proliferation such as cyclophosphamide (CTX), tyrosine kinase inhibitors such as sunitinib, sorafenib, imatinib, aclizumab, antibody including ADC against CD25, Denileukin Diftitox, IL-28B, IL28A and IL29.

Optionally agent that can inactivate MDSC (Myeloid-derived suppressor cell) can be added to the composition/formulation of the current inventions and those in U.S. patent application Ser. No. 15/945,741 to be injected intratumorally to treat cancer. Suitable amount can be between 0.001˜5% w/w. They can be in form of active molecule, prodrug, liposome, emulsion, micelle, insoluble precipitate (e.g. in complex with condensing agent), conjugated to polymer drug carrier (e.g. dextran), coated on or encapsulated in biodegradable micro particle/nano particle (e.g. those made of biodegradable polymer such as PLA, PLGA, PCL, PGA or PHB). Suitable size of the particle can be between 10 nm˜100 um. Examples of Myeloid-derived suppressor cell inactivating agent include Phosphodiesterase-5 inhibitors such as sildenafil and tadalafil, Nitro-aspirin (NO-aspirin) that interferes with MDSC nitric oxide metabolism, synthetic triterpenoids that deactivate MDSC by reducing reactive oxygen species (ROS), CSF-1R-blocking agents, Bardoxolone methyl (CDDO-Me), Cyclooxygenase 2 (COX2) inhibitors, arginase inhibitors such as N-hydroxy-L-Arginine (NOHA) and N(G)-Nitro-L-Arginine Methyl Ester (L-NAME), MDSC differentiating agents such as all-trans retinoic acid, 1α,25-hydroxyvitamin D3 and vitamin A, some cytotoxic agents that can cause MDSC depletion such as Gemcitabine, curcumin, docetaxel (DTX), STAT Tyrosine kinase inhibitors such as axitinib, sorafenib and sunitinib and combination of STAT inhibitor such as cucurbitacin with sialidase.

The intratumoral injection used in the current invention and those in U.S. patent application Ser. No. 15/945,741 can contain a viscosity enhancing agent to increase its viscosity after being injected, which acts as a sustained release formulation of both conjugate and immune enhancing agent. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipients such as hyaluronic Acid (linear or cross linked form), starch and carbomer. In some embodiments, the viscosity enhancing agent is biodegradable. The injection formulation can also be a thermal phase changing formulation. Thermal phase changing formulation is a formulation that change its phase from liquid at low temperature or room temperature (25 C) to semisolid/gel when temperature increases to body temperature (37 C), which can use poloxamer as excipient. A thermal phase changing injectable formulation containing both the conjugate or cancer killing microbes and immune enhancing agent such as TLR agonist can be injected intratumorally to treat cancer. The preparation of this kind of thermal phase changing injectable formulation can be adopted from related publications readily by the skilled in the art.

In one example, a solution containing 20˜200 mg/mL L-rhamnose-cholesterylamine conjugate of U.S. patent application Ser. No. 15/945,741, 3 mg/mL poly IC or 3 mg CpG ODN 2216 or both, 20 mg/mL biodegradable PLGA nano particles encapsulating 20% imiquimod, and granulocyte-monocyte colony-stimulating factor (10-200 μg/mL), L-Arginine, L-cysteine and L-tryptophan at 20˜100 mg/mL, poly aspirin 20 mg/mL, glutathione or SOD 5 mg/mL, N-hydroxy-L-Arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL is prepared. Suitable amount of surfactant can be added to from stable suspension. Suitable amount of carbomer is added to the solution to reach a viscosity of 5,000,000 cps. After the patient receive the intratumoral injection with the above formulation at 0.5 mL/cm3 tumor volume, the patient is intravenously injected with Ipilimumab 3˜10 mg/kg every 3 weeks for 4 doses, or Atezolizumab 1200 mg IV q3wk until disease progression.

U.S. patent application Ser. No. 15/945,741 by the current inventor disclosed native antigen-optional linker-cell surface anchoring molecule conjugate for cancer treatment and the use of it to treat cancer and compositions containing it. The current invention also disclose antibody binding molecule-optional linker-cell surface anchoring molecule conjugate and method using it to treat cancer and compositions containing it. In some embodiments the cancer cell inactivating agent can be cancer cell killing microbe or the combination of cancer cell killing microbe with said conjugate. The conjugate in the current invention and application Ser. No. 15/945,741 can be replaced with cancer cell killing microbes or their combination. The methods and compositions in the disclosure of current invention and prior application Ser. No. 15/945,741 can also use the cancer cell killing microbes instead of the conjugates or in combination of the cancer cell killing microbes. In the embodiments and examples of the current invention and prior application Ser. No. 15/945,741 the conjugate can be replaced with the cancer cell killing microbes or used in combination with the cancer cell killing microbes. Examples of cancer cell killing microbes can be either cancer cell killing bacterial or cancer cell killing virus (oncolytic virus) or cancer killing parasites or cancer killing fungi or any microbe that can kill cancer cells or their combination. They can be either given systematically or injected intratumorally. Examples of cancer cell killing bacterial include engineered Salmonella typhimurium described in doi: 10.1126/scitranslmed.aak9537, Clostridium novyi-NT spores described in DOI: 10.1126/scitranslmed.3008982, Salmonella typhimurium (VNP20009), Clostridium sporogenes, Coley's Toxins and BCG. Other bacterial that can kill the cancer cells when being injected into tumor can also be used. Example dose of cancer cell killing bacterial used for intratumoral injection can be between 100,000˜1000,000,000 copies for each tumor, e.g. 1-10 million C. novyi-NT or 10Λ8 CFU of Salmonella typhimurium can be injected to a tumor. Examples of cancer cell killing virus (oncolytic virus) include oncolytic poxvirus, JX-594, Imlygic (talimogene laherparepvec; T-VEC), enterovirus RIGVIR, oncolytic adenovirus(H101), Cavatak, oncolytic virus M1, CG0070, Reolysin et ac. More examples can be found at en.wikipedia.org/wiki/Oncolytic_virus. Example dose of cancer cell killing bacterial used for intratumoral injection can be between 10⁴˜10¹⁴ pfu for each tumor, e.g. 1×10⁹ pfu JX-594 can be injected into a tumor.

In one example, a solution containing 10Λ8 CFU of Salmonella typhimurium/mL, 3 mg/mL poly IC or 3 mg CpG ODN 2216 or both, 20 mg/mL biodegradable PLGA nano particles encapsulating 20% imiquimod is prepared. Optionally suitable amount of linear or cross linked hyaluronic acid is added to the solution as a viscosity enhancer to reach a viscosity of 5,000,000 cps. After the patient receive the intratumoral injection with the above formulation at 0.5 mL/cm3 tumor volume, the patient is intravenously injected with Ipilimumab 3˜10 mg/kg every 3 weeks for 4 doses, or Atezolizumab 1200 mg IV q3wk until disease progression.

In another example, a solution containing 1-10 million C. novyi-NT/mL, 3 mg/mL poly IC or 3 mg CpG ODN 2216 or both, 20 mg/mL biodegradable PLGA nano particles encapsulating 20% imiquimod, L-Arginine, L-cysteine and L-tryptophan at 20˜100 mg/mL, poly aspirin 20 mg/mL, glutathione or SOD 5 mg/mL, N-hydroxy-L-Arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL is prepared. Optionally suitable amount of hyaluronic acid is added to the solution to reach a viscosity of 5,000,000 cps. After the patient receive the intratumoral injection with the above formulation at 0.5 mL/cm3 tumor volume, the patient is intravenously injected with Ipilimumab 3˜10 mg/kg every 3 weeks for 4 doses, or Atezolizumab 1200 mg IV q3wk until disease progression.

In another example, a solution containing 1×10∧9 pfu JX-594/mL, 3 mg/mL poly IC or 3 mg CpG ODN 2216 or both, 20 mg/mL biodegradable PLGA nano particles encapsulating 20% imiquimod is prepared. Optionally suitable amount of hyaluronic acid is added to the solution to reach a viscosity of 5,000,000 cps. After the patient receive the intratumoral injection with the above formulation at 0.5 mL/cm3 tumor volume, the patient is intravenously injected with Ipilimumab 3˜10 mg/kg every 3 weeks for 4 doses, or Atezolizumab 1200 mg IV q3wk until disease progression.

In another example, a solution containing 1×10∧12 pfu oncolytic virus M1/mL, 3 mg/mL poly IC or 3 mg CpG ODN 2216 or both, 20 mg/mL biodegradable PLGA nano particles encapsulating 20% imiquimod, L-Arginine, L-cysteine and L-tryptophan at 20˜100 mg/mL, poly aspirin 20 mg/mL, glutathione or SOD 5 mg/mL, N-hydroxy-L-Arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL is prepared. Optionally suitable amount of hyaluronic acid is added to the solution to reach a viscosity of 5,000,000 cps. After the patient receive the intratumoral injection with the above formulation at 0.5 mL/cm3 tumor volume, the patient is intravenously injected with Ipilimumab 3˜10 mg/kg every 3 weeks for 4 doses, or Atezolizumab 1200 mg IV q3wk until disease progression.

In another example, the formulation is a solution containing 1×10∧9 pfu Imlygic/mL, 10 mg/mL CpG ODN 2216-fatty acid conjugate, 10 mg/mL imiquimod, 2 mg/mL poly IC, 10 mg/mL antibody against CD25, 10 mg/mL antibody against OX40, 1×10⁴-1×10⁵ U/mL of IFN-α, 1-10 MIU/mL IL-2. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression.

In another example, the formulation is a solution containing 20˜200 mg/mL L-rhamnose-cholesterylamine conjugate, 1×10∧9 pfu Imlygic/mL, 10 mg/mL CpG ODN 2216-fatty acid conjugate, 10 mg/mL imiquimod, 2 mg/mL poly IC, 10 mg/mL antibody against CD25, 10 mg/mL antibody against OX40, 1×10⁴-1×10⁵ U/mL of IFN-α, 1-10 MIU/mL IL-2. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression.

In one example, the formulation contains 1×10∧13 pfu oncolytic adenovirus(H101), 2 mg/mL imiquimod, 2 mg/mL poly IC, 5 mg/mL α-GalCer and 2 mg/mL neuraminidase (sialidase, human), etoricoxib or naproxen 10 mg/mL, L-Arginine and L-cysteine and L-tryptophan 10 mg/mL each, IL-28B 10 mg/mL, sorafenib 10 mg/mL, cytotoxic antibody against CD39 10 mg/mL, gemcitabine 10 mg/mL, cyclophosphamide 10 mg/mL in 1×PBS. Optionally suitable amount of hyaluronic acid is added to the solution to reach a viscosity of 5,000,000 cps. It can be injected into the tumor at 100˜500 uL/cm3 tumor volume to treat cancer every 10 days for total 3 times. Check point inhibitor can be given to the patient at the same time and later.

Antibody against the cancer cell killing microbes can also be given to the patient (e.g. IV injection) when cancer cell killing microbes is injected to the patient intratumorally. For example, when the said formulation described above containing oncolytic virus is injected to the tumor of the patient, antibody against the oncolytic virus protein that can be expressed on the cancer cell surface upon infection can be given to the patient to boost its anti cancer activity.

This can improve the tumor associated antigen presentation. For example, when HSV1716 is injected to the patients' tumor, humanized antibody against HSV1716 capsid can be iv injected to the patient at therapeutically effective amount.

In some embodiments, the cancer cell killing/inhibiting microbes (e.g. virus and bacterial) can also be engineered to express or produce or secret immune activity enhancing agent with recombination technology. Suitable immune activity enhancing agent can be selected from TLR agonist such as Bacterial lipoprotein including triacyl lipopeptides, Bacterial peptidoglycans as TLR 2 agonist, lipoteichoic acid, zymosan (Beta-glucan), heat shock proteins, Bacterial flagellin, profilin, bacterial diacyl lipopeptides, TLR peptide/protein agonist disclosed in patent applications WO2018055060A1, WO2013120073A1, WO2016146143A1 and US20180133295A1 and their citations, or their combinations. They can be either be expressed as membrane bound form or secreted form. Suitable immune activity enhancing agent can also be selected from granulocyte macrophage colony-stimulating factor, immunostimulatory monoclonal antibody, antibody for CD137, FMS-like tyrosine kinase 3 ligand (FLT3L), T-cell-tropic chemokines such as CCL2, CCL1, CCL22 and CCL17; B-cell chemoattractant such as CXCL13, Interferon gamma, type I IFN (e.g. IFN-a, IFN-beta); tumor necrosis factor (TNF)-beta, TNF-alpha, IL-1, Interleukin-2, IL-12, IL-6, IL-24, IL-2, IL-18, IL-4, IL-5, IL-6, IL-9, IL-28B and IL-13 or their derivatives, CD1d ligand, Vα14/Vβ8.2 T cell receptor ligand, iNKT agonist, antibody against OX 40, tumor necrosis factor, interferon gamma (IFNγ), Treg inhibitory agent such as inhibitory antibody against Treg (such as antibody against CD4, CD25, FOXP3 and TGF-(β or its receptor) or their combinations.

Furthermore, in some embodiments, the cancer cell killing/inhibiting microbes (e.g. virus and bacterial) can also be engineered to express or produce or secret enzymes that can produce anti cancer activity. Suitable enzyme can be selected from sialidase (e.g. bacterial sialidase such as V. cholerae sialidase or viral sialidase such as flu sialidase or animal sialidase or human sialidase), hyaluronidase (e.g. human recombinant Hylenex), adenosine deaminase (e.g. adenosine deaminase 2), peptide-N-glycosidase (e.g. PNGase F), b-N-Acetylglucosaminidase (e.g. recombinant from Streptococcus pneumonia), other endo-β-N-acetylglucosaminidases (Endo D and Endo H), exoglycosidases (such as β-galactosidase, neuraminidase and N-acetyl-β-glucosaminidase) and enzymes that can degrade mucin's carbohydrate part, as well as collagenase such as those from bacterial or human MMP No. 1, No. 8, No. 13, and No. 18.

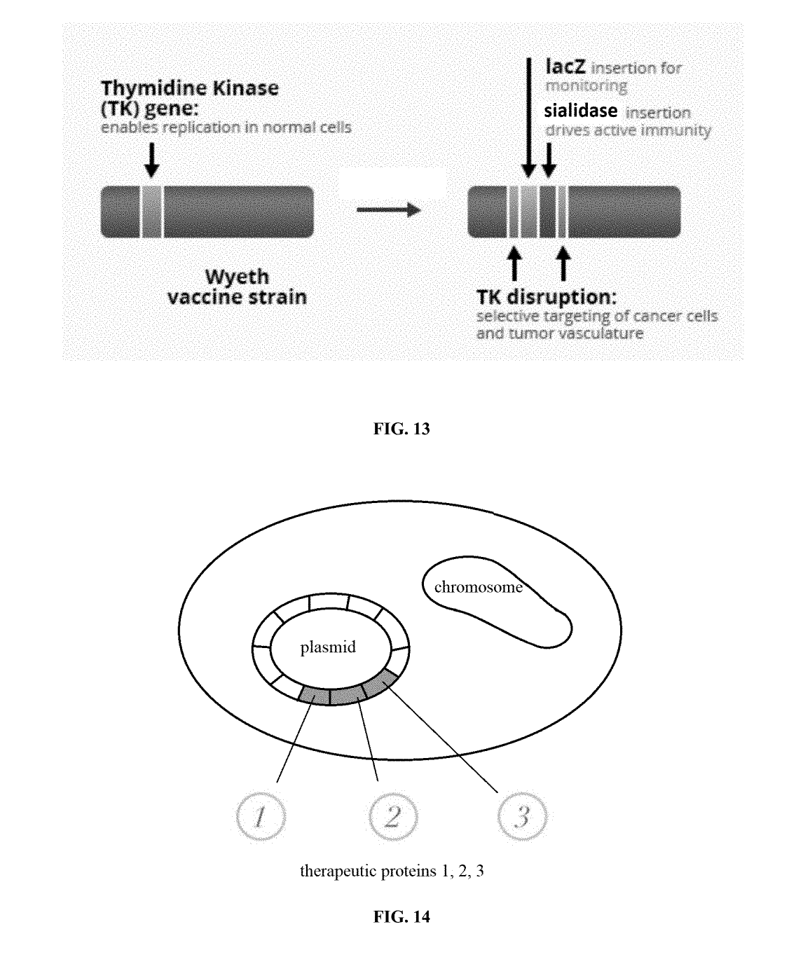

Engineering bacterial or virus to express the protein/peptide or enzyme listed above can be done easily with recombinatant technology by a skilled in the art. There are many protocols and formats in prior art publications that can be adapted for the current invention. For example, FIG. 13 shows an example of the construct of a JX-594 virus that can produce sialidase by replacing the GM-CSF sequence with a flu sialidase sequence in Pexa-Vec (JX-594) oncolytic virus.

In another example, WO2018006005A1 disclosed pseudotyped oncolytic viral delivery of therapeutic polypeptides. It described pseudotyped oncolytic viruses comprising nucleic acids encoding an engager molecule. In some embodiments, the pseudotyped oncolytic viruses comprise nucleic acids encoding an engager molecule and one or more therapeutic molecules. The current invention can simply use the sequence or sequences of the said enzymes and or protein/peptide of the current invention (e.g. TLR agonist peptide, sialidase and/or IL-2) as the therapeutic molecules in the pseudotyped oncolytic viruses of the prior art to construct the virus desired by the current invention. WO2017132552A1 disclosed oncolytic viral vectors and uses thereof. One can use the vector design to express the desired protein/peptide/enzyme of the current invention in a oncolytic virus to be used in the current invention.

Those protein/peptide/enzymes can also be easily incorporated into the plasmid of bacterial such as lactic acid bacterial to be expressed by it as shown in the figure below. FIG. 14 shows a construct of an anti cancer bacterial that produce 3 desired proteins such as those listed above.

For example, WO2016124239A1 disclosed recombinant probiotic bacteria for use in the treatment of a skin dysfunction, which express FGF2, IL4 and CSF1 by inserting nucleic acid sequence(s) encoding them. When these nucleic acid sequence(s) are replaced with nucleic acid sequence(s) of V. cholerae sialidase or collagenase clostridium histolyticum or adenosine deaminase 2 or N-acetyl-β-glucosaminidase or TLR peptide agonist, it becomes an embodiment of the current invention.

Previous U.S. patent application Ser. No. 16/004,415 from current inventor disclosed novel sialidase conjugate and the prozyme form of sialidase for cancer treatment. The sialidase in the conjugate or prozyme or fusion protein disclosed in Ser. No. 16/004,415 application and the sialidase in all the conjugates or prozymes or fusion proteins described in the current application including those descried later in the current invention can also be replaced by other enzyme selected from hyaluronidase (e.g. human recombinant Hylenex), adenosine deaminase (e.g. adenosine deaminase 2), peptide-N-glycosidase (e.g. PNGase F), b-N-Acetylglucosaminidase (e.g. recombinant from Streptococcus pneumonia), other endo-β-N-acetylglucosaminidases (Endo D and Endo H), exoglycosidases (such as (β-galactosidase and N-acetyl-β-glucosaminidase) and enzymes that can degrade mucin's carbohydrate part. Those novel conjugate or prozyme can be used to treat cancer. Prior U.S. patent application Ser. Nos. 15/169,640 and 15/373,483 from current inventor also disclose novel sialidase conjugate and the prozyme form of sialidase. The prozyme form of sialidase use sialidase inhibitor as masking moiety to block its activity before enzyme activation (e.g. tumor protease) in prior applications. An alternative is to use bulky steric hindrance masking moiety to block its activity before enzyme activation, similar to those described in the above said pro-antibody. The construct can be the same as the above pro-antibody except the antibody is replaced with sialidase.

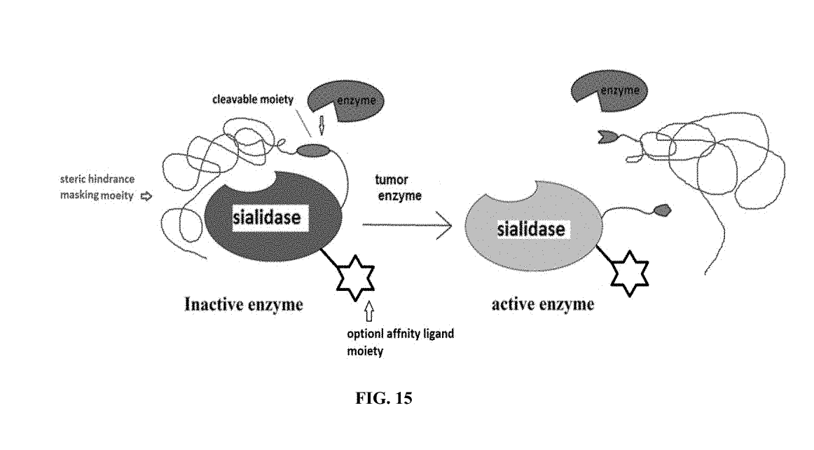

In some embodiments the masking moiety can be an enzyme inhibitor or a bulky high MW polymer that block the sialidase catalytic region by steric hindrance instead of enzyme inhibitor, or their combination. The masking moiety containing a large size moiety can be either synthetic polymer such as PEG (e.g. 10KD˜100KD PEG) or recombinant peptide (e.g. a polypeptide with MW 20KD˜200KD) such as the Xten peptide used in ProTia platform from Amunix or PAS peptide from XL-protein GmbH. The masking moiety is connected to a cleavable moiety, which is then connected to the sialidase. FIG. 15 show an example of steric hindrance based masking of sialidase to generate a sialidase prozyme. The affinity ligand for cell or pathogen surface maker can also be conjugated to sialidase. The conjugate can be either a fusion protein or synthesized with chemical conjugation. The fusion can be either at the N terminal or C terminal of sialidase.

The sialidase (either active form or prozyme form) can also be conjugated with one or more affinity ligand to the therapeutical antibody (e.g. an antibody against cancer cell such as Herceptin). It will bind to the therapeutical antibody and cleave the sialic acid on the cancer cells once the anti cancer antibody bind with cancer cells. This will provide targeted delivery of sialidase and increase therapeutical efficacy of the therapeutical antibody. It can be either pre-mixed with the therapeutical antibody to form the binding complex or injected to the patient separately to allow the sialidase-therapeutical antibody complex form in vivo. The affinity ligand can bind with either with Fc or Fab or F(ab′)₂ of the therapeutical antibody but should not block the binding of the therapeutical antibody to its target (non-neutralizing). Preferably the ligand binding with the therapeutical antibody should not inhibit the ADCC of antibody and should not inhibit complement activation. The therapeutical antibody binding ligand can either be peptide, antibody, antibody fragment, aptamer or small molecules. For example, when anti cancer therapeutical antibody is IgG containing humanized Fab, a non-neutralizing antibody or its F(ab′)₂ or Fab fragment against human IgG Fab region can be used to conjugate with sialic acid. In some embodiments, the antibody against human IgG Fab used for sialidase conjugation can be those used as secondary antibody against Fab in ELISA, for example, it can be Human IgG Fab Secondary Antibody (mouse anti human SA1-19255) from ThermoFisher or Mouse Anti-Human IgG Fab fragment antibody [4A11] (ab771) from Abcam or their F(ab)/Fab′/F(ab′)2 fragments. The anti-Human IgG Fab antibody or its fragment can be conjugated to the sialic acid via a linker (e.g. PEG or flexible peptide) either chemically or by expression. The sialidase can be either active sialidase or the prozyme form sialidase. For example, FIG. 11 in U.S. application Ser. No. 16/004,415 shows the scheme of this kind of sialidase. When the therapeutical antibody is an antibody against pathogens such as bacterial, the sialidase conjugate in the current invention can also be used to increase the efficacy of treating pathogens by removing the sialic acid on the pathogen surface. Another type of the novel sialidase derivative (e.g. sialidase conjugate) of the current invention is a conjugate of sialidase with affinity ligand (e.g. antibody) against immune cell surface marker, which will provide a sialidase based cancer immunotherapy as well as a method and reagent to improve the activity of immune activity against pathogen infection. The sialidase in the conjugate can be either the active enzyme form or the prozyme form. The conjugate can be constructed as a fusion protein by recombinant technology or by chemical conjugation (preferably by site specific conjugation).

Immune cells for the current invention include leucocytes such as neutrophils, eosinophils, basophils, lymphocytes, and monocytes. Example of them include B cells, T cells, NK cells, macrophage, neutrophil and dendritic cell.

The immune cell surface marker can be cluster of differentiation type surface protein of immune cells. Examples of the immune cell surface marker can be either cell type specific such as KIR, CD3, sialic acid-binding immunoglobulin-type lectins or shared by many different types of immune cells (e.g. MHC II or FcR such as Cd16a). The binding of ligands used for the conjugate preferably should have minimal negative impact on immune activity (cyto toxicity) of these cells against pathogens or cancer cells.

The immune cell surface marker can be the stimulatory checkpoint receptor such as CD 27, CD28, CD 80, CD86, CD40, CD137, OX40, GITR, ICOS and etc. The immune cell surface marker can also be the inhibitory checkpoint receptor such as A2AR, BTLA, CTLA-4, KIR, LAG3, PD-1, TIM-3, VISTA and etc. In some embodiments preferably the affinity ligand to conjugate sialidase should not activate the inhibitory checkpoint receptor. In some embodiments, the affinity ligand can also be ligand targeting cancer specific marker such as VEGF, EGF, EGFR, VEGFR, cancer surface marker (e.g. CD47, sialic acid or poly sialic acid, GD2), cancer cell produced inhibitory ligand that can bind with inhibitory checkpoint receptor such as PD-L1, B7-H3, B7-H4 and tec. Immuno cells are abundant in tumors. For example, Killer-cell immunoglobulin-like receptors (KIRs) is unique on NK cells and it inhibits NK cell activity similar to a check point inhibitor. Innate BMS is developing antibody against it to treat cancer (Lirilumab). Lirilumab or other antibody against KIRs can be conjugated with sialidase to treat cancer or pathogen infection. Other maker such as PD-1 and CTLA-4 on T cells can also be used. For example, FDA approved Ipilimumab (against CTLA-4) and Pembrolizumab (against PD-1) can be conjugated with sialidase. It is similar to bi-specific antibody format except one arm is antibody while another arm is enzyme instead of antibody. Anti PD-L1 antibody such as Atezolizumab can also be used to conjugate with sialidase. Besides these immune inhibitory cell surface markers, activating or non-regulatory marker such as CD3 on T cell or other markers on other immune cell can also be used. Non-antibody ligand such as peptide, aptamer or small molecule for these targets can also be used to conjugate with sialidase; there are several peptide and small molecule PD-1 or PD-L1 ligand available, which is disclosed in publications and literatures. For example, AUNP-12(Aur-012) peptide or those described in U.S. Patent application Ser. No. 2011/0318373 or the PD-1 or PD-L1 ligand from Aurigene Discovery Technologies or the D-Peptide Antagonist (disclosed in DOI: 10.1002/anie.201506225) can be readily adopted to conjugate with sialidase to be used in the current invention. The term conjugate here covers both chemical conjugation and fusion by expression methods. The sialidase conjugate in the current invention has the general formula as: sialidase-(optional linker)-affinity ligand. Examples of linker can be found in prior arts and many publications such as PEG or a flexible peptide such as a short Xten. The sialidase can be mutated to introduce coupling site for site specific conjugation. There are many site specific conjugation methods that can be readily adopted form prior arts, publications and literatures.

In one example, the sialidase-antibody against PD-1 (e.g. Pembrolizumab or the like) conjugate is prepared according to the protocol in Precision glycocalyx editing as a strategy for cancer immunotherapy, DOI: 10.1073/pnas.1608069113. The same conjugation protocol can be used except Pembrolizumab is used instead of Trastuzumab in the literature. In brief, Pembrolizumab bearing a C-terminal aldehyde tag is prepared. The functionalized antibody is first coupled to aminooxy-tetraethyleneglycol-azide (aminooxy-TEG-N3). Alternatively, NHS-PEG₅-azide can be used to introduce the azide linker to the antibody on its lysine site. Other site specific conjugation methods such as conjugating NH2-PEG₆-azide to antibody with mTgase can also be used. The protocol can be found in the prior arts and readily adopted for the current synthesis. Separately, V. cholerae sialidase is nonspecifically functionalized on lysine residues with bicyclononyne-N-hydroxysuccinimide ester (BCN-NHS). After an overnight reaction, excess linker is removed. Antibody adorned with the azide-functionalized linker is conjugated to BCN-functionalized V. cholerae sialidase via copper-free click chemistry. The desired conjugate is purified using a size-exclusion column. In another example, antibody against KIR (e.g. Lirilumab) is used instead of antibody against PD-1. In another example, antibody against Siglec-7 or siglec-9 is used instead. In another example, antibody or its fragment against sialic acid or hemagglutinin or lectin that can bind with sialic acid (e.g. Influenza hemagglutinin) is used instead. In another example, a D-peptide that bind with PD-L1: (D)NYSKPTDRQYHF (all amino acids are D amino acid in this peptide)—PEG₅-azide is conjugated to BCN-functionalized sialidase with click chemistry to generate a (D)NYSKPTDRQYHF—linker-sialidase conjugate. The antibody can also be replaced with its fragment such as Fab or F(ab′)2. Fusion protein can also be constructed instead of chemical conjugation. For example, FIG. 12 in U.S. application Ser. No. 16/004,415 shows examples of the fusion protein contain sialidase. The sialidase can be in prozyme form and the antibody can also be in probody or pro-antibody form or anti-cancer bi specific antibody form.

The sialidase in the above conjugates or prozymes can be replaced by other enzyme selected from hyaluronidase (e.g. human recombinant Hylenex), adenosine deaminase (e.g. adenosine deaminase 2), peptide-N-glycosidase (e.g. PNGase F), b-N-Acetylglucosaminidase (e.g. recombinant from Streptococcus pneumonia), other endo-β-N-acetylglucosaminidases (Endo D and Endo H), exoglycosidases (such as β-galactosidase and N-acetyl-β-glucosaminidase) and enzymes that can degrade mucin's carbohydrate part. Those novel conjugate or prozyme can be used to treat cancer.

Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. A cell surface anchoring conjugate comprising at least one antigen covalently bonded to a lipid moiety, or an analogue or derivative thereof, optionally via linker, wherein the antigen is a mimotope of a recombinant antibody.

2. The cell surface anchoring conjugate of claim 1, comprising more than one antigen.

3. The cell surface anchoring conjugate of claim 1, wherein the lipid moiety is selected from a sterol, 3β-cholesterylamine, cholesterol, a fatty acid, a triglyceride, a phospholipid, acetylated or non-acetylated glycerol, a sphingolipid, sphingosine, ceramide, a glycerolipid, a glycerophospholipid, glycerophosphoethanolamine and a steroid.

4. The cell surface anchoring conjugate of claim 1, wherein the linker is a peptide comprising one or more Lys, Arg, or other positively charged amino acid.

5. The cell surface anchoring conjugate of claim 1, wherein the recombinant antibody is a therapeutical antibody drug for cancer treatment.

6. The cell surface anchoring conjugate of claim 1, wherein the cell surface anchoring antigen conjugate further comprises a cancer cell binding domain, such as folic acid, RGD peptide, RGD peptidomimetic, or a TGF-α, GnRH, EGFR or VEGF antagonist.

7. A pharmaceutical composition comprising a cell surface anchoring conjugate and a Toll-like receptors (TLR) agonist, wherein the cell surface anchoring conjugate comprises a mimotope antigen of a recombinant antibody.

8. The pharmaceutical composition of claim 7, wherein the Toll-like receptors (TLR) agonist is CpG, poly IC, imiquimod, or a mixture thereof.

9. The pharmaceutical composition of claim 7, wherein the cell surface anchoring conjugate comprises a antigen moiety and a lipid moiety selected from a sterol, 3β-cholesterylamine, cholesterol, a fatty acid, a triglyceride, a phospholipid, acetylated or non-acetylated glycerol, a sphingolipid, sphingosine, ceramide, a glycerolipid, a glycerophospholipid, glycerophosphoethanolamine and a steroid.

10. The pharmaceutical composition of claim 7, wherein the mimotope antigen is a peptide antigen.

11. The pharmaceutical composition of claim 7, further comprises a recombinant antibody that can bind with the antigen.

12. A method of treating and/or inhibiting a tumor cell, comprising administering to a patient in need thereof a therapeutically effective amount of a mixture of the cell surface anchoring mimotope antigen conjugate and a Toll-like receptors (TLR) agonist together with a recombinant antibody that can binds with the antigen.

13. The method of claim 12, wherein the treating and/or inhibiting comprises preventing metastasis of the tumor.

14. The method of claim 12, wherein the method comprises administering a therapeutically effective amount of an immune check point inhibitor.

15. The method of claim 12, wherein the tumor is a melanoma.

16. The method of claims 12, wherein the administering is via intratumoral injection.
